(19) United States
(12) Patent Application Publication
(10) Pub. No.: US 2023/0402158 A1
(43) Pub. Date: Dec. 14, 2023

(54) VIRTUAL ASSISTANTS FOR PREEMPTIVE MEDICAL DATA ANALYSIS AND TREATMENT

(71) Applicant: AT&T Intellectual Property I, L.P., Atlanta, GA (US)

(72) Inventors: Eric Zavesky, Austin, TX (US); Zhengyi Zhou, Chappaqua, NY (US); Louis Alexander, Franklin, NJ (US); Jianxiong Dong, Pleasanton, CA (US); Qiong Wu, Bridgewater, NJ (US)

(21) Appl. No.: 17/806,197

(22) Filed: Jun. 9, 2022

(51) Int. Cl. G16H 40/20; G16H 10/60; G16H 70/40; G06F 9/451

(52) U.S. Cl. CPC G16H 40/20; G16H 10/60; G16H 70/40; G06F 9/453

(57) ABSTRACT

Virtual assistants (VAs) can be managed in interactions with users, other VAs, and devices, including in connection with medical care associated with users. A VA management component (VAMC) can analyze medical information and other information relating to users, including information obtained by a VA(s) from a user(s). Based on the analysis, VAMC can determine a model relating to medical care associated with users. VAMC can determine a proposed action relating to the health or medical care associated with a user based on the model. A VA can present action information relating to the proposed action, or other information relating to health or medical care associated with the user, to the user or a device associated with the user. VAMC can determine drug-drug or drug-food interactions or side effects, and the VA can notify the user regarding the drug-drug or drug-food interactions or side effects.

### TECHNICAL FIELD

This disclosure relates generally to electronic communications, e.g., to virtual assistants for preemptive medical data analysis and treatment.

### BACKGROUND

Communication devices (e.g., landline phones, mobile phones, electronic pads or tablets, computers, or other communication devices) can be utilized to engage in electronic communications (e.g., voice and/or data traffic) between entities associated with the communication devices. Virtual assistants (VAs) can be communication devices or can be part of or utilized by communication devices. Various services can be provided to and utilized by entities using VAs and/or communication devices in a communication network.

The above-described description is merely intended to provide a contextual overview regarding electronic communications, and is not intended to be exhaustive.

### BRIEF DESCRIPTION OF THE DRAWINGS

FIG. 1 illustrates a block diagram of an example system that can manage a VA(s) in interactions with users, in accordance with various aspects and embodiments of the disclosed subject matter.

FIG. 2 depicts a block diagram of a VA management component, in accordance with various aspects and embodiments of the disclosed subject matter.

FIG. 3 illustrates a block diagram of an example VA, in accordance with various aspects and embodiments of the disclosed subject matter.

FIG. 4 depicts a block diagram of an example VA system process that can manage collection of information associated with users, interactions with users (or other VAs or devices), analysis of information, determinations based on the analysis, proposed actions that can be taken or performed, and presentations of information to users (or other VAs or devices).

FIG. 5 depicts a block diagram of example communication device, in accordance with various aspects and embodiments of the disclosed subject matter.

FIG. 6 illustrates a flow chart of an example method that can manage a VA(s) in interactions with a user, including determining, and presenting to the user, information regarding a proposed action relating to medical care of the user, in accordance with various aspects and embodiments of the disclosed subject matter.

FIG. 7 presents a flow chart of an example method that can determine drug-drug interactions or side effects between drugs, and/or drug-food interactions or side effects between a drug and a food, in accordance with various aspects and embodiments of the disclosed subject matter.

FIG. 8 is a schematic block diagram illustrating a suitable operating environment.

### DETAILED DESCRIPTION

Various aspects of the disclosed subject matter are now described with reference to the drawings, wherein like reference numerals are used to refer to like elements throughout. In the following description, for purposes of explanation, numerous specific details are set forth in order to provide a thorough understanding of one or more aspects. It may be evident, however, that such aspect(s) may be practiced without these specific details. In other instances, well-known structures and devices are shown in block diagram form in order to facilitate describing one or more aspects.

Communication devices (e.g., landline phones, mobile phones, electronic pads or tablets, computers, devices in or integrated with vehicles, or other communication devices) can operate and communicate via wireline or wireless communication connections (e.g., communication links or channels) in a communication network to perform desired transfers of data (e.g., voice and/or data communications), utilize services, engage in transactions or other interactions, and/or perform other operations. Virtual assistants (VAs) (also referred to herein as virtual agents) can be communication devices or can be part of or utilized by communication devices. Various services, including medical and health related services, can be provided to and utilized by entities using VAs and/or communication devices in a communication network. For instance, in connection with providing a service, a communication session between communication devices, and/or between a communication device and an entity, can involve one or more human users, one or more VAs, and/or one or more other entities (e.g., software entities or components).

With regard to medical care of patients, as medical records are increasingly digitized and stored as electronic medical records, and as more and more new medical research is released, it can be challenging for medical care providers (e.g., physicians, medical care specialists, nurses, or other medical care providers) to stay desirably (e.g., sufficiently, suitably, or optimally) informed on the entire medical field, or even the entirety of a particular medical discipline, with regard to both new medical care and conflicts of existing medical care.

Also, there is a significant amount of medical information associated with patients that may be collected and evaluated by medical care providers. For instance, there are many in-home patient care options, and patient self-monitoring products and devices, that have been developed and utilized by patients at home or elsewhere, but often medical information and/or other pertinent information generated or derived from such options, products, and devices are not included in the data collection and evaluation by medical care providers. Further, with regard to predictive services, patients often have to share private data to private companies to receive advanced predictive services (e.g., DNA sequencing, option comparison, longevity evaluation, or other predictive services), but patients seldom are able to benefit from losing control of their own data associated with such predictive services, and such data often is not generally collected and evaluated by the medical care providers of those patients (outside of the limited evaluation of such data by the private companies).

Furthermore, cost overruns and estimates across different medical treatments and across different medical care providers have been, and continue to be, an undesirably murky and insufficiently explored solution. Complications between coordinating with insurance providers (e.g., health insurance provider, vehicle insurance provider, or other insurance provider), medical care providers, and a patient's personal nature can create undesirable struggles that can fall on the physician and/or the patient.

It can be desirable to overcome these and other deficiencies or inefficiencies associated with existing techniques or procedures associated with managing or providing medical care of patients.

To that end, techniques for managing VAs in interactions with users, other VAs, and devices, including in connection with medical care associated with the users, are presented. A VA management component (VAMC) can analyze medical information (e.g., EMRs and/or other medical information) and other information relating to users (e.g., patients), including information (e.g., medical information, including medical history, current or historical symptoms, personal information, user preferences, or other information) obtained by a VA(s) from a user(s), and/or relating to medicine (e.g., medical literature or journals, medical news, medical research or studies, or other medical information) generally. In some embodiments, the VAMC can employ artificial intelligence (AI)-based analysis, functions, techniques, models, or algorithms as part of the information analysis. Based at least in part on results of the analysis, the VAMC can determine a model relating to medical care associated with users, wherein the model can comprise respective user-specific models relating to medical care associated with respective users. With regard to a user, the VAMC can determine a proposed action relating to the health or medical care associated with the user based at least in part on the model or the user-specific model associated with the user. The VA can present action information relating to the proposed action, and/or other information relating to the health or medical care associated with the user, to the user or a device (e.g., communication device or other device) associated with the user.

During interactions between the VA (or VAMC) and a user or other entity or device, the VAMC and/or VA can desirably secure information (e.g., medical data, personal data, or other sensitive or private data) associated with a user from access by unauthorized entities or devices (e.g., the VAMC and/or VA can prevent unauthorized entities or devices from accessing or perceiving secured information associated with the user), and can control (e.g., control in a secure manner) the presentation or communication of secured information by the VA or VAMC to the user or other authorized entity or device, in accordance with defined VA management criteria, applicable data privacy laws and regulations (e.g., Health Insurance Portability and Accountability Act (HIPAA) or other applicable data privacy laws, and user data privacy rights, that apply to respective items of medical, personal, privatae, or sensitive data associated with users), and/or user preferences or information release or waiver authorizations issued or executed by the users.

With further regard to these and other aspects of the disclosed subject matter, in accordance with various embodiments, the VAs described herein can be or can comprise a medical-related VA that can be data driven by a patient's EMRs and other information relating to the patient or relating to medical care, wherein the VA(s) can automate or facilitate automating tasks associated with and/or on behalf of the patient, and/or automating tasks associated with and/or on behalf of medical care providers with regard to the patient, and improving medical staff productivity. The VA can represent each part, or at least desired parts, of patient care, and can intercommunicate with the patient, medical care providers, other VAs, or other devices, and/or can advocate on behalf of the patient. The VAMC and VAs described herein also can facilitate desirable (e.g., secure and authorized) data sharing (e.g., sharing of information relating to patients) between VAs, between a VA and a patient, between a VA and a medical care provider, between a VA and another device, and/or between a VA and another entity, and can provide or facilitate providing desirable (e.g., improved, suitable, or optimal) guided predictions (e.g., AI-based analysis and learning-guided predictions) and associated recommendations relating to medical care of patients based at least in part on analysis (e.g., AI-based analysis, learning, and models) of various types of medical information associated with patients and medical care, in accordance with the defined VA management criteria.

In accordance with various embodiments, the VAMC and associated VAs can provide closed and secure VA execution in a trusted medical environment, in accordance with the defined VA management criteria, the applicable data privacy laws and regulations, and/or user preferences or information release or waiver authorizations issued or executed by the users. The VAMC and associated VAs also can employ respective data-based triggers for respective persons (e.g., patients) to facilitate limiting execution costs and data privacy leakage.

In addition to analyzing EMRs and medical information (e.g., medical literature, medical studies, or other medical information) from data sources (e.g., medical journals, medical books, or other data sources), the VA and/or VAMC can incorporate information obtained from in-home patient care products or services, information obtained from patient self-monitoring products and devices, information obtained from predictive services (e.g., DNA sequencing, option comparison, longevity evaluation, or other predictive services), and/or other information related to patients into the analysis. Based at least in part on the results of such analysis (e.g., AI-based analysis, utilizing or applying an AI function or model), the VA and/or VAMC can determine and generate a model relating to medical care and medical conditions associated with users (e.g., patients), which can include respective user-specific models associated with (e.g., pertaining to) respective users. With regard to a user, the VA and/or VAMC can determine proposed actions relating to the medical care of the user based at least in part on the model or the user-specific model associated with the user. The VA can communicate or present action information relating to the proposed action(s) to the user or a communication device associated with the user. The proposed actions can comprise or relate to, for example, medical testing that the user can undergo or can be recommended to undergo, medical treatments or medical procedures for a medical condition that the user can undergo or can be recommended to undergo, a new or alternative medical treatment or medical procedure for a medical condition that the user can undergo or can be recommended to undergo (e.g., a different medical treatment or procedure of which the medical care provider(s) of the user is unaware), reminder or notification messages to remind the user of an upcoming medical testing, treatment, or procedure, diet or exercise recommendations to the user, diet or exercise reminder or notification messages to the user, or other proposed action relating to the medical care of the user.

In some embodiments, on behalf of a client (e.g., a patient or medical care provider), a VA can provide a second opinion (e.g., an on-demand second opinion) with regard to medical diagnosis, care, or treatment for a patient, or can perform recurring study or learning of recent literature (e.g., medical literature or studies) with regard to medical diagnoses, conditions, care, procedures, or treatments, without having explicit clinician review first. The VA also can determine and create follow-up actions for each person (e.g., actions to schedule and have medical testing, care, procedures, or treatments; actions to engage in exercise; or other types of actions), and/or can determine and present recommendations regarding specific physical examinations for a person, based at least in part on the results of analyzing current or historical medical data of the person (e.g., based at least in part on the user-specific model associated with the person).

The VA also can determine (e.g., calculate) and/or present information relating to medical treatment regimen, and/or costs or estimated costs associated with the medical treatment regimen, to a person (e.g., patient) without having to have undesirably lengthy or privacy exposing conversations with medical care providers or financial entities (e.g., insurance company representatives; or financing company representatives). The VA and/or the VAMC can, for example, formulate or facilitate formulating an entire path of the medical treatment regimen with desirable user transparency for the patient.

The VA and/or the VAMC can preemptively learn, identify, determine and/or test for drug-drug side effects or interactions between drugs, and/or drug-food side effects or interactions between food and a drug, based at least in part on the results of analyzing medical data associated with patients, drug data associated with drugs, food data associated with foods, and/or other desired information (e.g., medical, drug, and/or foods studies; news information relating to medicine, drugs, and/or foods). The VA can present (e.g., preemptively present) the patient with a notification message relating to, and notifying (e.g., warning) the patient of, any drug-drug side effects or interactions associated with one or more drugs, and/or any drug-food side effects or interactions associated with one or more drugs and foods. Such notification messages and/or other notifications relating to the medical care or treatment of a patient can enhance bedside manner for the patient and can allow contribution of external data to improve recurring medical care without the VA or patient having to first consult (and engage in a lengthy validation) with a clinician.

The techniques, VAMC, and VAs described herein can provide a number of advantages and benefits relating to providing information and services, including medical-related information and services, to users over existing techniques relating to providing information and services to users. The VA, as described herein, can automate the collection of information from a patient and retention (e.g., storage in a data store) of such information, which can provide an improved bedside manner for the user, in part, by enabling the patient to have the most impactful time with the patient's medical care provider (e.g., physician, medical specialist, nurse, clinician, or other medical care provider) when the patient has a medical visit or interaction with the medical care provider, instead of the patient having to repeatedly be subjected to the same questions and provide the same answers (and the medical care provider having to take the time to repeatedly ask the same questions or repeatedly discuss a medical diagnosis with the patient, or ask the same questions that other medical care providers have already asked the patient). The techniques, VAMC, and VAs described herein can enhance (e.g., increase or improve) confidence of the patient with regard to getting additional medical opinions and backup (e.g., supporting) information that can provide recommendations relating to medical care based at least in part on other cohort analysis (e.g., performed or received by the VA or VAMC). The techniques, VAMC, and VAs described herein also can enhance accuracy with regard to medical information and records, and other information, and can assist the patient in recalling specific times and events by precise recall (e.g., by the VA) of situation-specific desires (e.g., wants, needs, or requirements) of EMR details or other details associated with the patient. The techniques, VAMC, and VAs described herein also can provide improved transparency with regard to explanations of medical care process and medical care costs for the patient. The VAs described herein can enable a fast preview or consultation with or via a VA by a medical care provider before the medical care provider interacts with the patient (e.g., patient's physician can obtain information from the VA and/or can use the VA to consult with a medical expert, before interacting with the patient), which can extend or enable desirably fast diagnosis of a medical condition of the patient by the medical care provider, for desirably low-latency triage and assistance coordination that can improve patient care and outcomes. The techniques, VAMC, and VAs described herein further can provide enhanced (e.g., increased, improved, or optimized) privacy for closed-system operation and evaluation of new medical-related options and patient records.

These and other aspects and embodiments of the disclosed subject matter will now be described with respect to the drawings.

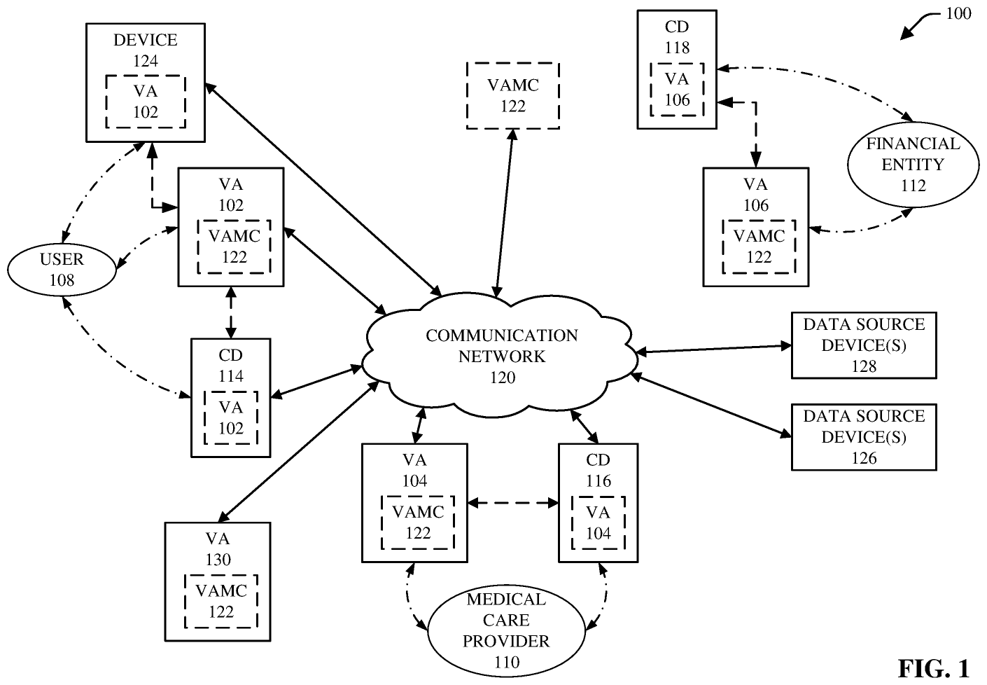

Referring now to the drawings, FIG. 1 illustrates a block diagram of an example system 100 that can manage a VA(s) in interactions with users (e.g., users, such as patients, physicians or other medical personnel, financial entities, or other users, and/or communication devices associated with the users), in accordance with various aspects and embodiments of the disclosed subject matter. The system 100 can comprise a desired number of VAs, such as VA 102, VA 104, and/or VA 106, that can be associated with various entities, such as a user 108 (e.g., patient or other user), a medical care provider 110 (e.g., physician, hospital, nurse, or other medical personnel or entity), and/or financial entity 112 (e.g., insurance company, financing company, or other financial entity). Each of the VAs (e.g., 102, 104, and/or 106) can interact with a user(s) (e.g., 108, 110, and/or 112) and can perform various tasks for or on behalf of the user(s) that can be associated with the VA. Some of the tasks can relate to the health, medical care, medical treatment, medical procedures, and/or other health or medical-related events associated with users (e.g., user 108), such as described herein.

In some embodiments, a VA (e.g., 102, 104, or 106) can be included in, integrated with, or implemented via a device (e.g., a VA device), which can be a type of communication device. In other embodiments, the VA can be included in, integrated with, or implemented via a communication device (e.g., a VA application installed on or accessed via the communication device), such as communication device (CD) 114, communication device 116, or communication device 118, associated with the user.

In accordance with various embodiments, the user (e.g., 108, 110, or 112) can interact directly with the VA (e.g., 102, 104, or 106) and/or device associated therewith, and/or the user can interact with the VA via the communication device (e.g., 114, 116, or 118) associated with the user, wherein the VA or device associated therewith can communicate with the communication device via a direct communication connection (e.g., audio or visual communication over the air, wireless communication connection, or wireline communication connection) or via a communication network 120. A communication device (e.g., 114, 116, or 118) can be, for example, a mobile and/or wireless communication device, such as a mobile phone, a landline or wireline phone, a device comprising a VA, a computer, a medical device or sensor, an electronic notebook, an electronic pad or tablet, an electronic gaming device, a personal digital assistant (PDA), electronic bodywear (e.g., electronic or smart glasses, electronic or smart watch), a set-top box, an Internet protocol (IP) television (IPTV), an Internet-of-Things (IoT) device, or other type of communication device that can operate and communicate in a communication network environment (e.g., an environment associated with the communication network 120).

In some embodiments, the communication network 120 can comprise a core network (e.g., a mobility core network) that can comprise one or more radio access networks (RANs) (not shown) that can comprise or be associated with a set of base stations (e.g., access points (APs)) (not shown) that can serve communication devices (e.g., VAs 102, 104, and/or 106; communication devices 114, 116, and/or 118; and/or other devices) located in respective coverage areas served by respective base stations in the communication network 120. In some embodiments, the RAN(s) can be a cloud-RAN (C-RAN) that can be located in or associated with a cloud computing environment, comprising various cloud network components of the communication network 120. The respective base stations can be associated with one or more sectors (not shown), wherein respective sectors can comprise respective cells. The cells can have respective coverage areas that can form the coverage area covered by the one or more sectors. One or more of the respective communication devices (e.g., VAs 102, 104, and/or 106; communication devices 114, 116, and/or 118; and/or other devices) can be communicatively connected to the communication network 120 via respective wireless communication connections with one or more of the respective cells.

In certain embodiments, the communication network 120 can comprise a packet-based communication network (e.g., IP packet-based communication network) that can facilitate wireline or wireless communication of data (e.g., data packets) between communication devices associated with the communication network 120 (e.g., the core network or packet-based communication network). The packet-based communication network can be associated with (e.g., communicatively connected to) the core network. One or more of the respective communication devices (e.g., VAs 102, 104, and/or 106; communication devices 114, 116, and/or 118; and/or other devices) can be communicatively connected to the communication network 120 via respective wireline or wireless communication connections.

It is to be appreciated and understood that, while the disclosed subject matter is typically described with regard to VAs, another type of software-based entity (e.g., software-based application or component) can be employed to perform the functions of the VAs, as described herein. It is to be appreciated and understood that, while some aspects of the disclosed subject matter are described where the user (e.g., 108, 110, or 112) can use the communication device (e.g., 114, 116, or 118) for communication (e.g., transmission, reception) of information (e.g., interaction-related and/or event-related information) to or from another device (e.g., another communication device, a VA, or other type of device), in certain aspects of the disclosed subject matter, the user can communicate information using, and can receive information from, the VA (e.g., 102, 104, or 106) (or associated device) (e.g., by speaking into an interface of or associated with the VA (or associated device), by receiving (e.g., hearing, viewing, or perceiving) information presented via an interface of or associated with the VA (or associated device)). For example, a VA (e.g., 102, 104, or 106) can be associated with (e.g., integrated with, attached to) a vehicle of the user (e.g., 108, 110, or 112), wherein the user can use (e.g., communicate using) the VA with regard to one or more services (e.g., health, exercise, and/or medical-related services, or other types of services) that can be provided using or facilitated by the VA. It further is to be appreciated and understood that a VA (e.g., 102, 104, or 106) exist on (e.g., be integrated with), accessed via, or otherwise associated with multiple devices (e.g., user 108 can access the VA 102 via a VA device, communication device 114, or another device).

A VA (e.g., 102, 104, or 106) can be a domain-specific VA or can be a generalized (or at least a more generalized) VA. For example, a domain-specific VA can be created and utilized to provide services or products for one or a relatively small subset of domains (e.g., a VA that provides or facilitates providing medical-related services or products; a VA that provides or facilitates providing food-related services or products; a VA that provides or facilitates providing video and/or audio content-related services or products; a VA that provides or facilitates providing sports-related services or products; or other type of domain-specific VA). As another example, a generalized (or a more generalized) VA can be created and utilized to provide services or products for all domains or at least a relatively larger subset of domains. The disclosed subject matter can enable the use of VAs to act as intermediaries and/or navigators for and on behalf of users and/or other entities, for example, with regard to interactions (e.g., medical or health-related interactions).

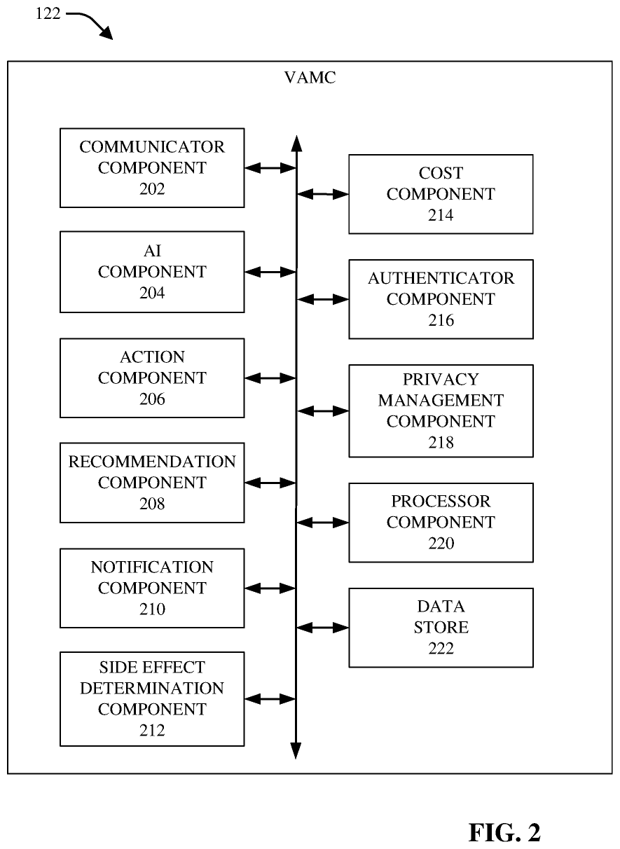

The system 100 can include a VA management component (VAMC) 122 that can manage (e.g., control) interactions and communications by the VAs (e.g., (e.g., 102, 104, or 106) with users or entities (e.g., 108, 110, or 112) and/or associated devices 124 (e.g., medical devices, sensors, or other devices) or communication devices (e.g., 114, 116, or 118), and/or between the VA (e.g., 102) and another VA (e.g., 104 or 106), in accordance with defined VA management criteria. Referring to FIG. 2 (along with FIG. 1), FIG. 2 depicts a block diagram of the VAMC 122, in accordance with various aspects and embodiments of the disclosed subject matter. In some embodiments, the VAMC 122 can be included in, integrated with, implemented via, or otherwise associated with the VA (e.g., 102, 104, or 106). In other embodiments, the VAMC 122 can be a stand-alone unit, can be part of another device or component, or can be distributed among various devices and components, wherein the VAMC 122 can be associated with (e.g., connected to) the VA (e.g., 102, 104, or 106) to facilitate communicating with and managing the VA.

The VAMC 122 and/or one or more VAs (e.g., 102, 104, or 106) can receive various types of data from various data sources. The VAMC 122 can comprise a communicator component 202 that can enable the VAMC 122 to receive or communicate information from or to other components or devices (e.g., VAs, communication devices, or other devices). In some embodiments, the VAMC 122 and/or one or more VAs (e.g., 102, 104, or 106) can receive medical data relating to health and medical care of users (e.g., 108), such as EMRs or other medical data, from medical care providers (e.g., 110) or entities (e.g., EMR archive or storage entities) associated with the medical care providers via communication device(s) (e.g., 116) or VA(s) (e.g., 104) associated with the medical care providers or associated entities. Such medical data can comprise personal data, demographic data (e.g., demographic data relating to demographic characteristics), and/or other data associated with the users.

The VAMC 122 and/or one or more VAs (e.g., 102, 104, or 106) also can receive health or medical related data from the users (e.g., patients, or family members or acquaintances of patients), such as user 108, via a VA (e.g., 102, 104, or 106), a communication device (e.g., 114 or 116), a device(s) 124 associated with the user, and/or another device. For instance, the VA(s) 102 associated with the user(s) 108 can receive information (e.g., medical data, including medical history (e.g., medical diagnosis or diagnoses, medical conditions, illnesses, diseases, allergies, medical treatments, medications, or operations or other medical procedures), current or historical symptoms; personal information; user preferences; or other information) associated with the user(s) 108 from the user(s) or another data source (e.g., another user or entity; communication device(s) 114; and/or device(s) 124), and the VA(s) 102 can provide (e.g., present or communicate) such information to the VAMC 122. The medical data also can include information relating to various types of drugs, including prescription drugs, over-the-counter drugs, or other types of drugs associated with the user(s) 108 (e.g., taken or utilized by the user 108), foods or products utilized by the user(s) 108, daily habits of the user(s) 108, and/or activities, exercising, or sports in which the user(s) 108 participates.

The VAMC 122 and/or one or more VAs (e.g., 102, 104, or 106) also can receive medical data, such as medical literature (e.g., medical books, medical journals), medical reports, medical studies, medical news, or other medical data from one or more data sources via one or more data source devices 126. The medical data also can comprise information (if any is available) that can be obtained from predictive services (e.g., DNA sequencing, option comparison, longevity evaluation, or other predictive services) via one or more data source devices, such as data source device 128.

For each user (e.g., user 108), the personal data can comprise or relate to the name, home address, residential status (e.g., home owner, or leasing a residence), employment, employment address, education, age, gender, religion, race, ethnicity, familial status, family medical history, or other personal data or personal characteristics associated with the user. For each user, the demographic data can comprise or relate to various demographic characteristics, such as, for example, residential characteristics (e.g., location or region of the residence, type of residence), age characteristics (e.g., age or age bracket), employment characteristics (e.g., type of employment), education characteristics (e.g., highest level of education, school or college attended), gender characteristics, religious characteristics, race characteristics, ethnicity characteristics, familial characteristics, or other demographic data or demographic characteristics associated with the user.

In certain embodiments, the VAMC 122 and/or one or more VAs (e.g., 102, 104, or 106) also can receive financial data or other data relating to medical care associated with users (e.g., patients), such as the user 108, from one or more financial entities, such as financial entity 112, or another data source associated with the one or more financial entities, via the VA 106, communication device 118, or other device. The financial data can relate to costs or estimated costs of medical care, medical treatment, medical procedures, or other medical or health-related events associated with the users (e.g., medical care, medical treatment, or medical procedures that a patient has received or may receive). The VAMC 122 and/or one or more VAs (e.g., 102, 104, or 106) also can receive other types of data that can relate to or impact the health or medical care of users (e.g., user 108). For instance, the VAMC 122 and/or one or more VAs (e.g., 102, 104, or 106) also can receive, from one or more data source devices (e.g., data source devices 126 and/or 128) associated with one or more data sources, food-related data relating to various foods to facilitate determining, learning, and/or understanding respective health impacts of respective foods on respective users, and/or respective drug-food interactions or side effects between respective drugs and respective foods.

The VAMC 122 or a VA can analyze the medical data, personal data, financial data, and/or other data associated with the users (e.g., 108) to facilitate determining proposed actions that can be taken by the users (e.g., 108), medical care providers (e.g., 110), financial entities (e.g., 112), or other entities, or by a VA (e.g., 102, 104, or 106), in connection with the health or medical care of the users. In some embodiments, as part of the analysis, the VAMC 122 can comprise an AI component 204 that can perform an analysis (e.g., an AI-based analysis) on the medical data, the financial data, and/or the other data, utilizing (e.g., applying) one or more AI-based techniques, functions, or algorithms, to enable the AI component 204 to learn from the medical data, financial data, or other data (e.g., learn respective data patterns in such data, learn about medical care, medical conditions, medical treatments, and medical procedures, learn interactions or side effects between drugs (e.g., medications or other drugs), learns interactions or side effects between drugs and foods (or other products), and/or learn other characteristics and features associated with the data). Based at least in part on the results of the analysis, the AI component 204 can determine, train, and generate a model that can relate to medical care and medical conditions associated with the users, wherein the model can model or be representative of the health and medical histories associated with the users, medical care and medical conditions associated with the users, medical knowledge database or library (e.g., database or library with knowledge of various medical care, diagnoses, treatments, procedures, or events), and/or other features relating to the users, medicine, or health. The AI component 204 can update (e.g., modify, adjust, or change), and further train and enhance, the model as additional data (e.g., medical data, drug data, food data, financial data, or other data) associated with users is received and analyzed by the AI component 204. In some embodiments, as part of the data analysis, and the determining and training of the model, the AI component 204 can employ and train Markov chains, a neural network(s) (e.g., can train the neural network(s)), or other AI-based or machine learning (ML) modeling, techniques, functions, or algorithms, such as more fully described herein.

The model can comprise respective user-specific models associated with respective users that can be based at least in part on the respective medical data associated with the respective users. For example, the AI component 204 can determine and generate a first user-specific model relating to the medical care and medical conditions associated with a first user (e.g., user 108), based at least in part on the results of analysis (e.g., performing an AI-based analysis) of the data, with a focus on or weighting of (e.g., higher weighting of) first data (e.g., first medical data, first financial data, first drug data, first food data, and/or other first data) associated with the first user; and can determine and generate a second user-specific model relating to the medical care and medical conditions associated with a second user, based at least in part on the results of analysis (e.g., performing an AI-based analysis) of the data, with a focus on or weighting of (e.g., higher weighting of) second data (e.g., second medical data, second financial data, second drug data, second food data, and/or other second data) associated with the second user.

For each user (e.g., user 108), based at least in part on the results of analyzing the model, which can include the user-specific model associated with the user, the VAMC 122 (e.g., employing an action component 206) or the VA (e.g., VA 102) can determine one or more proposed actions relating to the medical care associated with the user, wherein the proposed actions can comprise proposed actions that the VA can perform, the user can perform, or another entity can perform. The one or more proposed actions can comprise or relate to, for example, recommending that the user schedule a medical visit with the medical care provider (e.g., 110), scheduling the medical visit with the medical care provider for the user, notifying or reminding the user of the scheduled medical visit, recommending that the user exercise or perform certain exercises, reminding the user to exercise or perform certain exercises, notifying or reminding the user to take medication, notifying or warning the user about an irregular medical condition that has been detected (e.g., detected by the device(s) (e.g., 124) or health or medical application), providing the user with information (e.g., providing an explanation to the user and/or answering the user's questions) regarding a medical condition, medical care, medical treatment, or medical procedure, determining a cost or estimated cost of medical treatment or medical procedure, notifying the user of the cost or estimated cost of the medical treatment or medical procedure, determining a financing option(s) to finance payment of the medical care, treatment, or procedure, determining what portion of the cost of the medical care, treatment, or procedure is covered by a health insurance policy associated with the user, determining which health insurance policy is the most health and cost effective for the user when the user is obtaining or selecting a health insurance policy, identifying or learning about a new medical treatment or drug for a medical condition, obtaining a second opinion (e.g., from another VA or another medical care provider) regarding a medical condition or a course of medical treatment associated with the user, determining a drug-drug interaction or side effect between drugs, determining a drug-food interaction or side effect between a drug(s) and a food(s), notifying or warning the user of a drug-drug or drug-food interaction or side effect, and/or another proposed action relating to the health and medical care of the user.

The VA (e.g., VA 102) can perform the proposed action(s) (e.g., if the proposed action(s) is the type of action(s) the VA can perform) and/or present (e.g., display, communicate, or otherwise present) action information relating to the proposed action(s) to the user (e.g., user 108) or a device (e.g., communication device 114 or device 124) associated with the user. When the VA (e.g., VA 102) is presenting the action information to the user (e.g., 108), the user can perceive the action information presented by the VA (e.g., view the action information on a display screen of the VA, hear the action information via a speaker(s) of the VA, and/or experience or feel the action information, in a haptic form, via a haptic interface of the VA). If the VA has communicated the action information to a device (e.g., communication device 114 or device 124), the user can perceive the action information via the interfaces (e.g., display screen, audio speaker(s), earbuds or earphones, haptic interface, or another interface) of or associated with the device.

If the proposed action involves an action that the user can take or perform, the VA can monitor the activity of the user and can collect information relating to the activity of or associated the user (e.g., monitor and track the user exercising, monitor and track the user taking medication, and/or being informed by the user or the device 124 that the user is exercising or taking medication, monitoring or tracking body and health conditions of the user, monitor and track that the user went to the medical visit at the medical care provider, monitor and track that the user has undergone medical testing, treatment, or procedure, or monitor and track other activity of or associated with the user). The VA (e.g., 102) can communicate the information relating to the activity of or associated with the user to the VAMC 122, and the VAMC 122 (e.g., employing the AI component 204) can update the model based at least in part on the results of analyzing (e.g., performing an AI-based analysis of) the information relating to the activity of or associated with the user along with historical information (e.g., medical data, personal data, or other data) associated with the user and/or other users.

The following are some non-limiting examples of the VAs (e.g., 102, 104, and/or 106) and VAMC 122 utilizing the model, including a user-specific model associated with the user 108, to facilitate determining proposed actions relating to the health and medical care of the user 108 that can be taken or performed by a VA (e.g., VA 102), user 108, or the device 124, and/or making other determinations relating to the health and medical care of the user 108, in accordance with the defined VA management criteria. The AI component 204 can determine and generate the model based at least in part on the historical data (e.g., historical medical data, personal data, financial data, and/or other data) associated with the users, including the user 108, and/or current data (e.g., current medical data, personal data, financial data, and/or other data) associated with the users, including the user 108, wherein the AI component 204 can analyze and incorporate the current data, along with the historical data, into the model to update the model.

In some embodiments, prior to, and in connection with, an upcoming medical visit with the medical care provider 110, the VA 102 can query the user 108 and/or collect information (e.g., medical data, personal data, financial data, or other data) from the user 108, wherein such information can be pertinent to the medical visit. For instance, the querying of the user 108 and/or the information collected before the medical visit by the VA 102 can be the types of queries a medical care provider 110 may ask the user 108 in connection with the medical visit and the types of information the medical care provider 110 can desire to obtain from the user 108 in connection with the medical visit.

As an example of utilizing the model to facilitate determining proposed actions and/or making other determinations, based at least in part on analysis (e.g., AI-based analysis) of the model, including the user-specific model associated with the user 108, the VA 102 or VAMC 122 can determine a medical condition (e.g., heart-related condition, brain-related condition, lung-related condition, kidney-related condition, cancer-related condition, pregnancy, or other medical condition) that the user 108 has or at least potentially may have, and/or can determine medical care, medical treatment, medical testing and/or medical procedure (e.g., blood testing, lung testing, blood glucose testing, heart testing, operation, heart-related procedure, biopsy, X-ray, magnetic resonance imaging (MRI), computed tomography (CT) scan, dental procedure, eye-related procedure, or other medical testing or medical procedure), and/or medication that the user 108 can be recommended or directed to have in connection with the medical condition. The VA 102 can present information relating to the medical condition, and/or the medical care, medical treatment, medical testing and/or medical procedure and/or medication, to the user 108 via an interface(s) of the VA 102, the communication device 114, or device 124 for consideration or action by the user 108, in accordance with the defined VA management criteria. In some embodiments, the VA 102 (e.g., employing recommendation component 208) can present a recommendation message to the user 108 via an interface(s) associated with the VA 102, the communication device 114, or device 124, wherein the recommendation message can recommend that the user 108 undergo the determined medical care, medical treatment, medical testing, and/or medical procedure, and/or take the determined medication, in accordance with the defined VA management criteria.

As another example, based at least in part on analysis (e.g., AI-based analysis) of the model, including the user-specific model associated with the user 108, the VA 102 or VAMC 122 can determine exercises or a form of exercising (e.g., walking, jogging, calisthenics, sports, swimming, weight lifting, yoga, or other type of exercise) that can be recommended to the user 108 to perform to facilitate improving the health of the user 108 and/or to facilitate alleviating, mitigating, or preventing a medical condition or issue associated with, or that may be associated with (e.g., may be experienced by), the user 108. The VA 102 can present information relating to the exercises or form of exercising, and/or a recommendation to perform such exercises or form of exercising, to the user 108 via an interface(s) associated with the VA 102, the communication device 114, or device 124 for consideration or action by the user 108, in accordance with the defined VA management criteria.

In some embodiments, at or near the time (e.g., at or within a defined amount of time before) the VA 102 has scheduled such exercising for the user 108, the VA 102 (e.g., employing notification component 210 can present a reminder or notification message to the user 108 to remind or notify the user 108 to perform the exercise(s) or form(s) of exercising at the scheduled time (or other desired time). In certain embodiments, the VA 102 can determine a desirable (e.g., suitable or optimal) time to schedule such exercising for the user 108 based at least in part on the model, the type(s) of exercising to be performed by the user 108, the availability of any exercise equipment associated with such exercising, and/or the user's schedule (e.g., schedule relating to work, family, friend, or other commitments, social events, or other events), wherein the VA 102 can determine the user's schedule based at least in part on an electronic calendar (e-calendar) of the user 108 and/or feedback information received from the user 108 (e.g., VA 102 can ask user 108 when is a good time(s) for the user 108 to exercise).

In still other embodiments, the VA 102 can determine instructions relating to the exercise(s) to be performed by the user 108, and can present such instructions to the user 108 via an interface(s) of the VA 102, the communication device 114, or device 124, wherein the instructions can instruct or teach the user 108 how to properly perform the exercise(s) in a desirable (e.g., useful, beneficial, healthy, safe, or optimal) manner, in accordance with the defined VA management criteria. In yet other embodiments, if, based at least in part on the model and/or feedback from the user 108, the VA 102 determines that an alternative exercise or an alternative way of performing an exercise can be desirable (e.g., preferable, suitable, or more appropriate) to the user 108 due in part to the health or medical condition of the user 108, the VA 102 or VAMC 122 can determine the alternative exercise or the alternative way of performing an exercise, and the VA 102 (e.g., employing the recommendation component 208 of the VAMC 122) can present information relating to the alternative exercise or the alternative way of performing an exercise to the user 108, via an interface(s) associated with the VA 102, the communication device 114, or device 124, to advise the user 108, recommend to the user 108, and/or provide instruction to the user 108 with regard to the alternative exercise or the alternative way of performing an exercise, in accordance with the defined VA management criteria.

In certain embodiments, if, based at least in part on analysis (e.g., AI-based analysis) of the model and environmental conditions (e.g., weather conditions, allergen conditions, or other environmental conditions) associated with an area (e.g., outdoor area, such as a park, outdoor pool, city streets, or other area) where the user 108 is scheduled to exercise or may exercise, the VA 102 determines that the user 108 performing the exercising in the area under the environmental conditions can be undesirable (e.g., unsuitable, suboptimal, or unwanted) to the user 108, the VA 102 can determine an alternate time or alternate place (e.g., indoors or in a different area) for the user 108 to exercise, and/or an alternative form of exercising for the user 108 to perform, to avoid the undesirable exercising conditions. For instance, if the VA 102 determines that, in the area at the exercise time, the weather is going to be too hot, too cold, too humid, rainy, otherwise undesirable to the user 108, and/or certain allergens in the area are undesirably high and may cause the user 108 to have an undesirable allergic reaction (e.g., sneezing, headache, difficulty in breathing, or other allergic reaction), the VA 102 can determine a desirable alternate time or alternate place for the user 108 to exercise, and/or a desirable alternative form of exercising for the user 108 to perform, to avoid the undesirable exercising conditions. The VA 102 (e.g., employing the recommendation component 208 or notification component 210 of the VAMC 122) can present, to the user 108 (e.g., via an interface(s) of the VA 102, the communication device 114, or device 124), a notification or recommendation message relating to the undesirable environmental conditions in the area that may render exercising in the area undesirable to the user 108, and/or recommending the desirable alternate time or alternate place for the user 108 to exercise, and/or the desirable alternative form of exercising for the user 108 to perform, in accordance with the defined VA management criteria. The user 108 can take the information in such notification or recommendation message under consideration in determining where and when to exercise, and/or what type of exercise to perform.

As still another example, based at least in part on analysis (e.g., AI-based analysis) of the model, including the user-specific model associated with the user 108, the VA 102 or VAMC 122 (e.g., employing a side effect determination component 212 of the VAMC 122) can determine drug-drug interactions or side effects between two or more drugs. For instance, drug-related data analyzed by the VAMC 122 (e.g., the AI component 204 of the VAMC 122) in connection with determining the model can comprise data relating to a first group of ingredients of a first drug, a second group of ingredients of a second drug, medical literature, medical research, or medical studies relating to the first drug and second drug, news information relating to news about the first drug and second drug, and/or other desired information relating to the first drug and second drug. Based at least in part on the AI-based analysis of the data, including the drug-related data, and/or the health or medical characteristics of the user 108, and as part of the training of the model, the AI component 204 can learn about the first drug and second drug, including learning about, inferring, or determining drug-drug interactions or side effects between the first drug and second drug. In some instances, a drug-drug interaction or side effect can be something that can be experienced by virtually any person who uses the first drug and second drug. In other instances, a particular drug-drug interaction or side effect can be something that typically can be experienced by a person who has certain health or medical characteristics and who uses the first drug and second drug. As part of the learning and training of the model, the AI component 204 can learn, infer, or determine whether there is a drug-drug interaction or side effect that can be experienced by virtually any person who uses the first drug and second drug, and/or can learn, infer, or determine whether there is a drug-drug interaction or side effect that typically can be experienced by a person who has certain health or medical characteristics and who uses the first drug and second drug.

If the VA 102 or VAMC 122 determines that there is the drug-drug interaction or side effect associated with (e.g., between) the first drug and second drug in relation to the use of such drugs by the user 108, the VA 102 (e.g., employing the recommendation component 208 or notification component 210 of the VAMC 122) can present information relating to the drug-drug interaction or side effect associated with the first drug and second drug to the user 108, via an interface(s) associated with the VA 102, the communication device 114, or device 124, to notify or warn the user 108 of such drug-drug interaction or side effect, recommend that the user 108 not take the first drug and/or the second drug, and/or recommend that the user 108 contact the medical care provider 110 regarding such drug-drug interaction or side effect (e.g., before the user 108 stops taking the first drug or second drug, in case the medical care provider 110 may tell the user 108 to not stop taking the first drug or second drug, for some medical reason), in accordance with the defined VA management criteria. Additionally or alternatively, the VA 102 (e.g., employing the recommendation component 208 or notification component 210 of the VAMC 122) can present the information relating to the drug-drug interaction or side effect to the VA 104 or communication device 116 associated with the medical care provider 110, and the VA 104 or communication device 116 (e.g., via an interface(s) associated with the VA 104 or the communication device 116) can present the information relating to the drug-drug interaction or side effect to the medical care provider 110, to notify or warn the medical care provider 110 of such drug-drug interaction or side effect, recommend to the medical care provider 110 that the user 108 not take the first drug and/or the second drug, and/or recommend to that the medical care provider 110 contact the user 108 regarding such drug-drug interaction or side effect, in accordance with the defined VA management criteria.

As yet another example, based at least in part on analysis (e.g., AI-based analysis) of the model, including the user-specific model associated with the user 108, the VA 102 or VAMC 122 (e.g., employing the side effect determination component 212 of the VAMC 122) can determine drug-food interactions or side effects between a drug(s) and a food(s). For instance, drug-related data and food-related data analyzed by the VAMC 122 (e.g., the AI component 204 of the VAMC 122) in connection with determining the model can comprise data relating to a first group of ingredients of a drug, a second group of ingredients of a food, medical literature, medical research, or medical studies relating to the drug and food, news information relating to news about the drug and food, and/or other desired information relating to the drug and food. Based at least in part on the AI-based analysis of the data, including the drug-related data and food-related data, and/or the health or medical characteristics of the user 108, and as part of the training of the model, the AI component 204 can learn about the drug and food, including learning about, inferring, or determining drug-food interactions or side effects between the drug and the food. In some instances, a drug-food interaction or side effect can be something that can be experienced by virtually any person who uses (e.g., takes or ingests) the drug and the food. In other instances, a particular drug-food interaction or side effect can be something that typically can be experienced by a person who has certain health or medical characteristics and who uses the drug and the food. As part of the learning and training of the model, the AI component 204 can learn, infer, or determine whether there is a drug-food interaction or side effect that can be experienced by virtually any person who uses the drug and the food, and/or can learn, infer, or determine whether there is a drug-food interaction or side effect that typically can be experienced by a person who has certain health or medical characteristics and who uses the drug and the food.

If the VA 102 or VAMC 122 determines that there is the drug-food interaction or side effect associated with (e.g., between) the drug and the food in relation to the use of such drug and food by the user 108, the VA 102 (e.g., employing the recommendation component 208 or notification component 210 of the VAMC 122) can present information relating to the drug-food interaction or side effect associated with the drug and the food to the user 108, via an interface(s) associated with the VA 102, the communication device 114, or device 124, to notify or warn the user 108 of such drug-food interaction or side effect, recommend that the user 108 not utilize or consume the drug and/or the food, and/or recommend that the user 108 contact the medical care provider 110 regarding such drug-food interaction or side effect (e.g., before the user 108 stops taking the drug or consuming the food, in case the medical care provider 110 may tell the user 108 to not stop taking the drug, for some medical reason), in accordance with the defined VA management criteria. Additionally or alternatively, the VA 102 (e.g., employing the recommendation component 208 or notification component 210 of the VAMC 122) can present the information relating to the drug-food interaction or side effect to the VA 104 or communication device 116 associated with the medical care provider 110, and the VA 104 or communication device 116 (e.g., via an interface(s) associated with the VA 104 or the communication device 116) can present the information relating to the drug-food interaction or side effect to the medical care provider 110, to notify or warn the medical care provider 110 of such drug-food interaction or side effect, recommend to the medical care provider 110 that the user 108 not take the drug and/or the food, and/or recommend to that the medical care provider 110 contact the user 108 regarding such drug-food interaction or side effect, in accordance with the defined VA management criteria. In certain embodiments, the VA 102 or VAMC 122 (e.g., employing the side effect determination component 212 of the VAMC 122) can perform a similar analysis and make determinations regarding any drug-product interactions or side effects between a drug(s) and a product(s) (e.g., ingestible product, topical product, or other product).

As still another example, there may be instances where the VA 102, VA 104, and medical care provider 110 are not aware of a certain medical condition, medical treatment, medical procedure, or medication because the certain medical condition, medical treatment, medical procedure, or medication is relatively new and/or not well known throughout the medical community or by others. In such instances, one or more VAs (e.g., VA 102 and/or VA 104) can collaborate with one or more other VAs, such as VA 130, wherein the one or more other VAs (e.g., VA 130) can have, or at least potentially can have, information or access to information relating to the certain medical condition, medical treatment, medical procedure, or medication. The VA 102 and/or VA 104, based at least in part on the model and querying or interaction between the VA 102 and/or VA 104 and the one or more other VAs, such as VA 130, the VA 102 or VA 104, or VAMC 122, can learn, infer, or determine that the user 108 has the certain medical condition that was previously unknown to the VA 102 and VA 104 (e.g., can diagnose or provide a preliminary diagnosis that the user 108 has the certain medical condition, and/or can determine a certain medical treatment or medical procedure that the user 108 can receive or undergo, or a certain medication that that the user 108 can take, to treat a medical condition that the user 108. The VA 102 (e.g., employing the recommendation component 208 or notification component 210 of the VAMC 122) can present information relating to the certain medical condition, medical treatment, medical procedure, or medication to the user 108 via an interface(s) of the VA 102, the communication device 114, or device 124, in accordance with the defined VA management criteria. Additionally or alternatively, the VA 104 (e.g., employing the recommendation component 208 or notification component 210 of the VAMC 122) can present the information relating to the certain medical condition, medical treatment, medical procedure, or medication to the medical care provider 110 via an interface(s) of the VA 104 or the communication device 116, in accordance with the defined VA management criteria.

In some embodiments, if the VA 102, VA 104, or medical care provider 110 has made a preliminary determination regarding diagnosing the user 108 with a medical condition and/or a medical treatment, medical procedure, or medication that is to be recommended for treatment of a medical condition the user 108 has been diagnosed with, the one or more other VAs (e.g., VA 130) can be queried by or interacted with (e.g., collaborated with) by the VA 102 or VA 104 to obtain a second opinion from the one or more other VAs (e.g., VA 130) with regard to diagnosis of a medical condition for the user 108, and/or a medical treatment, medical procedure, or medication that is to be recommended or utilized for treatment of a medical condition of the user 108, based at least in part on the respective models and/or the respective knowledge databases associated with (e.g., available to or accessible by) the one more other VAs (e.g., VA 130), in accordance with the defined VA management criteria. The VA 102 or VA 104 can respectively present information relating to the second opinion to the user 108 or medical care provider 110 for consideration and/or action with regard to the medical care of the user 108, in accordance with the defined VA management criteria.

In certain embodiments, the various VAs (e.g., VAs 102, 104, and/or 130) can collaborate with each other to vote on whether the user 108 has a certain medical condition or on what medical condition the user 108 has, or whether a certain medical treatment, medical procedure, or medication is to be recommended to treat a medical condition for which the user 108 has been diagnosed, based at least in part on the respective models and/or the respective knowledge databases associated with (e.g., available to or accessible by) the respective VAs (e.g., VAs 102, 104, and/or 130). For instance, the medical condition that has the most votes by the VAs relative to the vote totals associated with the other medical conditions can be determined (e.g., by the VA 102, 104, or 130, or VAMC 122) to be the medical condition with which the user 108 can be diagnosed; or the medical treatment, medical procedure, or medication that has the most votes by the VAs relative to the vote totals associated with the other medical treatments, medical procedures, or medications can be determined (e.g., by the VA 102, 104, or 130, or VAMC 122) to be the medical treatment, medical procedure, or medication that is to be recommended or utilized for treatment of the user 108.

As yet another example, based at least in part on analysis (e.g., AI-based analysis) of the model, including the user-specific model associated with the user 108, the VA 102 or VAMC 122 (e.g., employing a cost component 214 of the VAMC 122) can determine a cost (e.g., actual cost, estimated cost, and/or net cost after insurance company, government agency, or other entity pays a portion of the cost) of medical care, medical treatment, medical procedure, and/or medication that the user 108 is going to receive or that the user 108 has been recommended to receive (e.g., by the VA 102, VAMC 122, or medical care provider 110). As part of the analysis of the model and determining the cost, the VA 102 or VAMC 122 can take into account the various costs of various items of the medical care, medical treatment, medical procedure, and/or medication, insurance coverage or other financial funding (e.g., government funding, private funding, or other funding) or discounts that can be applied to reduce the cost on one or more of such items to the user 108, length of treatment of the user 108, and/or additional or incidental costs (e.g., cost of travel to and from medical care providers, cost of time off of work, or other additional or incidental costs). If there are different courses of action (e.g., different courses of medical care, medical treatment, medical procedure, and/or medication), different medical care providers that can provide portions of the medical care, medical treatment, medical procedure, and/or medication, different types of insurance coverage or different financial funding that can be available depending in part on the course of action taken, the VA 102 or VAMC 122 (e.g., employing the cost component 214) can determine the respective costs associated with the respective courses of action, based at least in part on the results of analyzing the model. The VA 102 (e.g., employing the recommendation component 208 or notification component 210 of the VAMC 122) can present, to the user 108 (e.g., via an interface(s) of the VA 102, the communication device 114, or device 124), a notification or recommendation message that can comprise information relating to the cost of the medical care, medical treatment, medical procedure, and/or medication, and/or the respective costs of the respective courses of action relating to the medical care of the user 108, in accordance with the defined VA management criteria. The user 108 can take the information in such notification or recommendation message under consideration in determining what course of action (which also can include declining such medical care) the user 108 desires to take with regard to medical care for the user 108.

In accordance with various embodiments, the VAs (e.g., 102, 104, 106, and/or 130) and VAMC 122 can utilize desirable (e.g., suitable, secure, efficient, reliable, or optimal) authentication, data security, and data privacy techniques, procedures, and algorithms, in accordance with the defined VA management criteria. For instance, the VAMC 122 can comprise an authenticator component 216 that can authenticate users attempting to utilize or access a VA (e.g., 102, 104, 106, and/or 130) or the VAMC 122, and/or access or retrieve information (e.g., private or sensitive medical data, personal data, or other private or sensitive data) of users that is maintained (e.g., stored) by or can be accessed by the VA or VAMC 122. The authenticator component 216 can utilize single-factor or multi-factor authentication, username and password-based authentication procedures and algorithms, biometric (e.g., fingerprint or handprint, eye or iris-related biometric information, facial biometric information, voice biometric information, or other biometric information) authentication procedures and algorithms, certificate-based authentication procedures and algorithms, token-based authentication procedures and algorithms, security questions, and/or another desired authentication procedures and algorithms Applying one or more desired authentication techniques, procedures, and algorithms, the authenticator component 216 can receive authentication information (e.g., authentication credential, token, or other information) from a user or entity (e.g., another VA, a communication device or other type of device, or other human or non-human entity) that is attempting to access the VA or VAMC 122, and can analyze the received authentication information and stored authentication information (e.g., stored in the VA or VAMC) associated with a user or entity account associated with the VA or VAMC 122.

If, based at least in part on the results of analyzing the received authentication information and the stored authentication information, the authenticator component 216 determines that the received authentication information does not satisfy a defined matching criterion (e.g., does not match or does not sufficiently match) with respect to the stored authentication information, the authenticator component 216 can deny authentication of the user or entity, and can deny the user or entity access to the VA and VAMC 122, and information associated therewith. In some embodiments, if the authenticator component 216 does not authenticate the user or entity due to a failure of the user or entity to provide authentication information that satisfies the defined matching criterion, the authenticator component 216 can allow the user or entity one or more additional authentication attempts, up to a defined maximum number of authentication attempts, to provide valid authentication information associated with the user or entity account, in accordance with the defined VA management criteria. If the define maximum number of authentication attempts is reached without valid authentication information being received from the user or entity, the authenticator component 216 can lock out the user or entity from further attempts at authenticating with the VA or VAMC 122 and gaining access to the VA or VAMC 122 and information stored therein or associated therewith until the lock associated with the user account is removed by the authenticator component 216 (e.g., after a defined amount of time or indefinite amount of time has elapsed and/or one or more other conditions for unlocking are determined to be satisfied).

If, instead, based at least in part on the results of analyzing the received authentication information and the stored authentication information, the authenticator component 216 determines that the received authentication information satisfies the defined matching criterion (e.g., matches or at least sufficiently matches) with respect to the stored authentication information, the authenticator component 216 can authenticate the user or entity (e.g., can determine that the user or entity is authenticated). The VAMC 122 can comprise a privacy management component 218 that can grant the user or entity a group of access rights to access the VA and VAMC 122, and certain information stored therein or associated therewith, in accordance with the group of access rights, based at least in part on the valid authentication information, applicable data privacy laws (e.g., HIPAA or other data privacy laws, and user data privacy rights, that apply to respective items of medical, personal, or sensitive data associated with users), respective user preferences or authorizations associated with respective users, and/or the defined VA management criteria (which can be based at least in part the applicable data privacy laws and the user preferences). For instance, the privacy management component 218 can determine what access rights (e.g., what access rights can be included the group of access rights) that a user or entity can be permitted to have, in accordance with the valid authentication credentials, the applicable data privacy laws, the respective user preferences or authorizations associated with the respective users, and/or the defined VA management criteria. The privacy management component 218 can grant respective (e.g., different) groups of access rights, and, accordingly, respective (e.g., different) levels of access to the VA and VAMC 122, and the information stored therein or associated therewith (e.g., respective portions of the information), to respective authenticated users or entities based at least in part on the type of user or entity (e.g., patient, medical care provider that is treating the patient, medical personnel who is not treating the patient, another VA, a communication device or other device, or other type of entity), respective valid authentication information provided by respective users or entities and/or respective authentications of a user or entity (e.g., different authentication credentials can be associated with different levels of access to the VA and VAMC 122, and data stored therein or associated therewith), respective user preferences or authorizations associated with the respective user accounts or items of information (e.g., a user preference or signed authorization of the user (e.g., user 108) that indicates or specifies that the user is desiring and/or authorizing the release of all or certain portions of the medical, personal, or sensitive data associated with the user), in accordance with the valid authentication credentials, the applicable data privacy laws, the respective user preferences or authorizations associated with the respective users, and/or the defined VA management criteria.

For example, with a regard to a user (e.g., user 108), such as a patient, associated with a user account, the privacy management component 218 can grant a first group of access rights to the user (e.g., user 108) associated with the user account that can allow or enable the user to access medical, personal, or sensitive data associated with the user account (e.g., data in the EMRs or personal records associated with the user) and certain other information, in accordance with the first group of access rights and the applicable data privacy laws. With regard to a medical care provider 110 (e.g., physician or hospital, and other authorized medical personnel associated therewith) that is provider medical care to the user (e.g., user 108), the privacy management component 218 can grant a second group of access rights to the medical care provider 110, wherein the second group of access rights can allow or enable the medical care provider 110 to access medical, personal, or sensitive data associated with the user account (e.g., data in the EMRs or personal records associated with the user) and some other information (e.g., other medical, personal, or sensitive data associated with other user accounts associated with other patients of the medical care provider 110; business information associated with the medical care provider 110; or some other types of information), in accordance with the second group of access rights and the applicable data privacy laws. In some embodiments, the VA 104 associated with the medical care provider 110 can have the same group of access rights as the medical care provider 110, and, in other embodiments, the VA 104 can have a different group of access rights than that of the medical care provider 110.

With regard to another medical care provider that is not currently providing, or has not previously provided, medical care to the user (e.g., user 108), the privacy management component 218 can grant a third group of access rights to this other medical care provider, wherein the third group of access rights can allow or enable the medical care provider 110 to access certain information (e.g., certain medical, personal, or sensitive data associated with other user accounts associated with patients of that other medical care provider; business information associated with that other medical care provider; or some other types of information), but does not grant such other medical care provider access to the medical, personal, or sensitive data associated with the user account associated with the user (e.g., user 108) (unless the user has signed an authorization to grant access to this other medical care provider, or has otherwise permitted this other medical care provider to access, the medical, personal, or sensitive data associated with the user account), in accordance with the third group of access rights and the applicable data privacy laws.

With regard to the VA 102, which can be associated with (e.g., owned or operated by) the user 108, the privacy management component 218 can grant a fourth group of access rights to the VA 102, wherein the fourth group of access rights can allow or enable the VA 102 to access medical, personal, or sensitive data associated with the user account (e.g., data in the EMRs or personal records associated with the user) and various other items of information (e.g., other data associated with the user 108), in accordance with the fourth group of access rights and the applicable data privacy laws. With regard to another VA (e.g., VA 130) that is not associated with the user 108, the privacy management component 218 can grant a sixth group of access rights to the other VA, wherein, if the user 108 has signed authorization for the user's medical, personal, and sensitive data associated with the user account to be shared with the other VA, the sixth group of access rights can allow or enable the other VA to access the medical, personal, or sensitive data associated with the user account and/or certain other items of information (e.g., other data associated with the user 108 that is not subject to the applicable data privacy laws), in accordance with the sixth group of access rights and the applicable data privacy laws; or, if the user 108 has not authorized the user's medical, personal, and sensitive data associated with the user account to be shared with the other VA, the sixth group of access rights can be more limited and may only allow the other VA to certain items of information associated with the user 108 that are not subject to the applicable data privacy laws, while not allowing the other VA to access the medical, personal, or sensitive data associated with the user account, in accordance with the sixth group of access rights and the applicable data privacy laws. As a result, if the user 108 has not authorized the VA 102 or medical care provider 110 (or associated VA 104) to share the medical, personal, or sensitive data associated with the user account of the user 108 with the other VA (e.g., VA 130), the privacy management component 218 can secure the medical, personal, or sensitive data associated with the user account (e.g., can enforce data security rules with respect to such data) to prevent the other VA from accessing such data from the VA 102 (or medical care provider 110 or VA 104) and prevent the VA 102 (or medical care provider 110 or VA 104) from sharing such data with the other VA.

In some embodiments, the privacy management component 218 can apply or associated respective tags (e.g., privacy tags) with respective items of data (e.g., medical data; personal data; sensitive data; non-medical, non-personal, and non-private data; or other type of data) stored in or accessible to the VA (e.g., 102, 104, 106, and/or 130) or VAMC 122, wherein respective tags can be associated with respective levels of data privacy and/or can indicate the respective levels of privacy applicable to the respective items of data. The privacy management component 218 can store the respective tags with the data (e.g., in the data store 222) or a different storage location (e.g., in the data store 222 or another data store), and can map or otherwise associate the respective tags with the respective items of data. The privacy management component 218 can determine which items of data that a user or entity is permitted to access or retrieve based at least in part on the respective tags associated with the respective items of data and the group of access rights granted to the user or entity.

In certain embodiments, the privacy management component 218 can determine what information associated with the user 108 can be presented to the user 108 via an interface(s) of the VA 102, based at least in part on the context associated with the VA 102 and the user 108, in accordance with user preferences of the user 108, data release authorization (if any) signed by the user 108, the applicable data privacy laws, and/or the defined VA management criteria. The privacy management component 218 can determine the context associated with the VA 102 and the user 108, based at least in part on the results of analyzing various factors (e.g., characteristics, attributes), comprising conditions (e.g., environmental and other conditions) associated with the VA 102 and user 108, the interaction between the VA 102 and the user 108, feedback information received from the user 108, another interaction (if any) between the user 108 or the VA 102 and another entity (e.g., another person (if any) in the same location as the user 108 or VA 102), the interface(s) (e.g., interface(s) of the VA 102 or interface(s) of the communication device 114 or device 124) available to the user 108 or currently being used by the user 108, the type of information that is intended to be presented by the VA 102 to the user 108, and/or another factor.

For example, if, based at least in part on the results of analyzing the various factors, the privacy management component 218 determines that the context is that the user 108 is alone in a room at home where the VA 102 (and/or the communication device 114 or device 124) also is located, the privacy management component 218 can determine that the VA 102 can utilize the display screen, audio speakers, or other desired (e.g., suitable) interface of the VA 102 (or interface of the communication device 114 or device 124, if determined to be in the same room) to present information, including medical, personal, or sensitive information associated with the user 108, to the user 108. If, instead, based at least in part on the results of analyzing the various factors, the privacy management component 218 determines that the context is that the user 108 is not alone in the user's present location where the VA 102 (and/or the communication device 114 or device 124) also is located (e.g., there is one or more other persons at that location), the privacy management component 218 can determine that the VA 102 only can utilize a certain (e.g., a secure or private) interface(s) (e.g., earbuds or earphones) associated with (e.g., communicatively connected to) the VA 102 (or associated with the communication device 114 or device 124) to present secured information (e.g., medical, personal, or sensitive information) associated with the user 108 to the user 108, unless the user 108 provides an authorization (e.g., signs or electronically signs an information release or waiver) to communicate the secured information to the user 108 via another interface(s) (e.g., display screen, audio speakers, or other interface of or associated with the VA 102, communication device 114, or device 124).

In some embodiments, if the user 108 is determined to not be alone, the VA 102 (e.g., employing the privacy management component 218 of the VAMC 122) can present the user 108 an electronic authorization or waiver form (e.g., on the display screen or via audio presentation of such form) prior to, and in connection with, the anticipated presentation of the secured information associated with the user 108. If the user 108 (after being authenticated by the authenticator component 216) signs or otherwise authorizes (e.g., electronically signs or otherwise authorizes) the presentation of the secured information by the VA 102 to allow the VA 102 to utilize another interface(s) associated with the VA 102 (or the communication device 114 or device 124) to present the secured information to the user 108, the VA 102 can present the secured information via the desired (e.g., suitable or optimal) interface(s) associated with the VA 102 (or the communication device 114 or device 124).

Additionally or alternatively, if the user 108 is determined to not be alone, the VA 102 (e.g., employing the notification component 210 and the privacy management component 218 of the VAMC 122) can notify the user 108 that the VA 102 has secured information associated with the user 108 that the VA 102 wants to present to the user 108, and can prompt the user 108 to utilize a certain interface(s) (e.g., a display screen of the VA 102 (or communication device 114 or device 124), earbuds associated with the VA 102 (or communication device 114 or device 124), or other interface) to access, receive, or perceive (e.g., view or listen to) the secured information and/or authorize the presentation of the secured information, via the certain interface(s), to the user 108.

In certain embodiments, if the user 108 is determined to not be alone (and if no authorization for the sharing or release of the secured information has been received from the user 108), the VA 102 (e.g., as managed by the privacy management component 218 of the VAMC 122) can present non-secured information (e.g., general non-secured information, or non-secured information associated with the user 108) to the user 108 via a desired interface(s) associated with the VA 102 (or communication device 114 or device 124) (e.g., in accordance with user preferences of the user 108), and can present the secured information associated with the user 108 via the certain (e.g., the secure or private) interface(s) (e.g., earbuds or earphones) associated with (e.g., communicatively connected to) the VA 102 (or associated with the communication device 114 or device 124).

With further regard to the AI component 204, the AI component 204 can perform an AI and/or ML-based analysis on data, such as medical data associated with users (e.g., user 108), personal data associated with users, medical data relating to medical care, medical conditions, medical treatments, and medical procedures, drug-related data relating to drugs (e.g., medications or other drugs), food-related data relating to foods, product-related data relating to products, medical information (e.g., medical information from medical literature or journals, medical research or studies, medical reports, medical-related news, or other medical information from other data sources), data relating to medical care providers (e.g., medical care provider 110), financial data associated with users, financial data relating to medical care, information relating to data privacy laws, and/or other desired data, such as more fully described herein. In connection with or as part of such an AI or ML-based analysis, the AI component 204 can employ, build (e.g., construct or create), and/or import, AI and/or ML techniques and algorithms, AI and/or ML models (e.g., trained models), neural networks (e.g., trained neural networks), Markov chains (e.g., trained Markov chains), and/or graph mining to render and/or generate predictions, inferences, calculations, prognostications, estimates, derivations, forecasts, detections, and/or computations that can facilitate determining or learning data patterns in data, determining or learning about medical diagnoses or medical conditions associated with users (e.g., patients), determining or learning about medical care, medical treatments, and medical procedures associated with (e.g., that can be recommended to or provided to) users (e.g., patients), determining or learning medical testing that a user can undergo or can be recommended to undergo, determining or learning medical treatments or procedures for a medical condition that the user can undergo or can be recommended to undergo, determining or learning a new or alternative medical treatment or medical procedure for a medical condition that the user can undergo or can be recommended to undergo (e.g., a different medical treatment or procedure of which the medical care provider(s) of the user is unaware), determining a reminder or notification message to present to a user to remind the user of an upcoming medical testing, treatment, or procedure, determining or learning diet or exercise recommendations that can be presented to the user, determining or learning interactions or side effects between drugs and foods (or other products), determining data that can be presented in accordance with data privacy laws, determining a form of presentation and/or an interface(s) by which to present data in accordance with data privacy laws, determining or learning other characteristics and features associated with the data, and/or automating one or more functions or features of the disclosed subject matter, as more fully described herein.

Based at least in part on the results of the analysis, the AI component 204 can determine, train, and generate a model that can relate to health, medical care, medical conditions, medical treatment or procedures, and/or medications associated with the users, wherein the model can model or be representative of the health and medical histories associated with the users, medical care and medical conditions associated with the users, medical knowledge database or library (e.g., database or library with knowledge of various medical care, diagnoses, treatments, procedures, or events), and/or other features relating to the users (e.g., user 108), medicine, or health. The AI component 204 can update (e.g., modify, adjust, or change), and further train and enhance, the model as additional data (e.g., medical data, drug data, food data, financial data, or other data) associated with users is received and analyzed by the AI component 204. In some embodiments, as part of the data analysis, and the determining and training of the model, the AI component 204 can employ (and/or train) Markov chains, a neural network(s), or other AI-based or ML-based modeling, techniques, functions, or algorithms.

The AI component 204 can employ various AI-based or ML-based schemes for carrying out various embodiments/examples disclosed herein. In order to provide for or aid in the numerous determinations (e.g., determine, ascertain, infer, calculate, predict, prognose, estimate, derive, forecast, detect, compute) described herein with regard to the disclosed subject matter, the AI component 204 can examine the entirety or a subset of the data (e.g., data in or associated with users, medicine, drugs, foods, products, data privacy laws, user preferences, or other data) to which it is granted access and can provide for reasoning about or determine states of the system and/or environment from a set of observations as captured via events and/or data. Determinations can be employed to identify a specific context or action, or can generate a probability distribution over states, for example. The determinations can be probabilistic; that is, the computation of a probability distribution over states of interest based on a consideration of data and events. Determinations can also refer to techniques employed for composing higher-level events from a set of events and/or data.

Such determinations can result in the construction of new events or actions from a set of observed events and/or stored event data, whether or not the events are correlated in close temporal proximity, and whether the events and data come from one or several event and data sources. Components disclosed herein can employ various classification (explicitly trained (e.g., via training data) as well as implicitly trained (e.g., via observing behavior, preferences, historical information, receiving extrinsic information, and so on)) schemes and/or systems (e.g., support vector machines, neural networks, expert systems, Bayesian belief networks, fuzzy logic, data fusion engines, and so on) in connection with performing automatic and/or determined action in connection with the claimed subject matter. Thus, classification schemes and/or systems can be used to automatically learn and perform a number of functions, actions, and/or determinations.

A classifier can map an input attribute vector, z=(z1, z2, z3, z4, . . . , zn), to a confidence that the input belongs to a class, as by f(z)=confidence(class). Such classification can employ a probabilistic and/or statistical-based analysis (e.g., factoring into the analysis utilities and costs) to determinate an action to be automatically performed. A support vector machine (SVM) can be an example of a classifier that can be employed. The SVM operates by finding a hyper-surface in the space of possible inputs, where the hyper-surface attempts to split the triggering criteria from the non-triggering events. Intuitively, this makes the classification correct for testing data that is near, but not identical to training data. Other directed and undirected model classification approaches include, e.g., naïve Bayes, Bayesian networks, decision trees, neural networks, fuzzy logic models, and/or probabilistic classification models providing different patterns of independence, any of which can be employed. Classification as used herein also is inclusive of statistical regression that is utilized to develop models of priority.

In accordance with various embodiments, the VAMC 122 can comprise a processor component 220 that can work in conjunction with the other components (e.g., communicator component 202, AI component 204, action component 206, recommendation component 208, notification component 210, side effect determination component 212, cost component 214, authenticator component 216, privacy management component 218, and data store 222) to facilitate performing the various functions of the VAMC 122. The processor component 220 can employ one or more processors, microprocessors, or controllers that can process data, such as information relating to users, medical care providers, VAs, communication devices or other devices, interactions, events, contexts of users or interactions, status or progress of interactions, medicine, medical care, medical treatment, medical procedures, drugs, foods, products, proposed actions relating to medical care associated with users, costs associated with medical care, health insurance or other insurance, demographic data, data privacy, authorizations to authorize sharing or releasing secured information (e.g., medical, personal, or other sensitive data associated with users, such as patients), access rights to access data, activities relating to interactions, environmental conditions associated with users or interactions, conversations associated with participants of interactions, identifiers or authentication credentials associated with entities, devices, or components, voice generation of a VA, characteristics or modulations of one or more voices generated by a VA, updates to user profiles of users, parameters, traffic flows, policies, defined VA management criteria, algorithms (e.g., VA management algorithm(s), AI-based algorithms, or other algorithms), protocols, interfaces, tools, and/or other information, to facilitate operation of the VAMC 122, as more fully disclosed herein, and control data flow between the VAMC 122 and other components (e.g., VAs, communication devices, devices, base stations, network devices of the communication network, data sources, applications, or other component or device) or users associated with the VAMC 122.

The data store 222 that can store data structures (e.g., user data, metadata), code structure(s) (e.g., modules, objects, hashes, classes, procedures) or instructions, information relating to users, medical care providers, VAs, communication devices or other devices, interactions, events, contexts of users or interactions, status or progress of interactions, medicine, medical care, medical treatment, medical procedures, drugs, foods, products, proposed actions relating to medical care associated with users, costs associated with medical care, health insurance or other insurance, demographic data, data privacy, authorizations to authorize sharing or releasing secured information (e.g., medical, personal, or other sensitive data associated with users, such as patients), access rights to access data, activities relating to interactions, environmental conditions associated with users or interactions, conversations associated with participants of interactions, identifiers or authentication credentials associated with entities, devices, or components, voice generation of a VA, characteristics or modulations of one or more voices generated by a VA, updates to user profiles of users, parameters, traffic flows, policies, defined VA management criteria, algorithms (e.g., VA management algorithm(s), AI-based algorithms, or other algorithms), protocols, interfaces, tools, and/or other information, to facilitate controlling operations associated with the VAMC 122. In an aspect, the processor component 220 can be functionally coupled (e.g., through a memory bus) to the data store 222 in order to store and retrieve information desired to operate and/or confer functionality, at least in part, to the communicator component 202, AI component 204, action component 206, recommendation component 208, notification component 210, side effect determination component 212, cost component 214, authenticator component 216, privacy management component 218, processor component 220, data store 222, and/or other component of the VAMC 122, and/or substantially any other operational aspects of the VAMC 122.

In some embodiments, if the VAMC 122 is remote from a VA (e.g., VA 102, VA 104, VA 106, or VA 130) and manages the VA (e.g., VAMC 122 provides a central management service that can manage the VA), and connectivity between the VA and VAMC 122 is not available for a period of time or is otherwise not suitable (e.g., poor connectivity between the VA and VAMC 122), the VA can continue to operate to interact with a user(s) (user 108, and/or other users, VAs, devices, or entities), wherein the VA can have virtually full functionality (e.g., almost the same functionality as when the VA is connected to the VAMC 122), such as more fully described herein, or at least limited functionality (e.g., a portion of its full functionality), that can be utilized to interact with the user(s) (user 108 and/or other users, VAs, devices, or entities). In certain embodiments, the VA can comprise or be associated with a local VAMC that can perform all, virtually all, or at least a desirable portion of the functions (e.g., VA management functions, data analysis functions, operational functions, or other functions) that the VAMC 122 can perform, as more fully described herein, and can manage operation of the VA in a same or similar manner that the VAMC 122 can manage the VA, for example, when the VAMC 122 is not connected to the VA, and/or can coordinate and/or share management of the VA with the VAMC 122 when the VAMC 122 is connected to the VA.

During the time that the VA (e.g., VA 102, VA 104, VA 106, or VA 130) is operating using the local VAMC (e.g., when the VAMC 122 is not connected, or at least not suitably connected, to the VA), the local VAMC can monitor an interaction between the VA and the user(s) (user 108 and/or other users, VAs, devices, or entities), and can collect and store (e.g., in a data store) information relating or relevant to the interaction (e.g., dialog of the interaction, sensor data, or other desired information). The local VAMC can utilize and analyze such information to determine how to manage, and to manage, operation of the VA during the interaction in a same or similar manner as the VAMC 122 can manage the VA when connected to the VAMC 122, as more fully described herein.

When the VA (e.g., VA 102, VA 104, VA 106, or VA 130) is able to connect (e.g., suitably connect) with the VAMC 122, the local VAMC can synchronize with the VAMC 122 to exchange information with the VAMC 122, including the information relating or relevant to the interaction (or any other interaction associated with the VA) during the time when the VA and VAMC 122 were not connected, and/or other information that the VAMC 122 desires to communicate to the VA or local VAMC. For example, as part of the local VAMC synchronizing with the VAMC 122, the local VAMC can communicate, to the VAMC 122, information regarding dialog that occurred (e.g., between the user 108 and VA) during the interaction, sensor data collected by the VA or local VAMC, analysis data or results (e.g., analysis data or results from data analysis performed by the local VAMC or VA), user sentiment, user personality attributes, VA personality attributes, and/or environmental conditions, or other desired information.

In some embodiments, the VAMC 122 and/or one or more VAs (e.g., VA 102, VA 104, VA 106, VA 130, or other VA), individually or collectively (e.g., the VAMC 122 and one or more VAs; or two or more VAs), can perform various tasks concurrently, simultaneously, substantially simultaneously, or in parallel. For example, a VA (e.g., VA 102) can perform multiple tasks (e.g., data analysis, collecting information, presentation of information, or other tasks) concurrently, simultaneously, substantially simultaneously, or in parallel. The VAMC 122 also can perform multiple tasks concurrently, simultaneously, substantially simultaneously, or in parallel.

With further regard to the communication network 120 depicted in FIG. 1, the communication network 120 can comprise one or more wireline communication networks and one or more wireless communication networks, wherein the one or more wireless communication networks can be based at least in part on one or more various types of communication technology or protocols, such as, for example, 3G, 4G, 5G, or x generation (xG) network, where x can be virtually any desired integer or real value; Wi-Fi; Gi-Fi; Hi-Fi; or other communication technology or protocol. The communication network 120 (e.g., a core network, cellular network, or a network comprising a core network, cellular network, and/or an IP-based network) can facilitate routing voice and data communications between a communication device(s) (e.g., VAs (e.g., 102, 104, 106, and/or 130), communication devices (e.g., 114, 116, and/or 118), devices (e.g., 124, 126, and/or 128, VAMC 122, IoT device, or other communication device) and another communication device (e.g., another of such communication devices) associated with the communication network 120 in the communication network environment. The communication network 120 (e.g., core network or IP-based network of the communication network 120) also can allocate resources to the communication devices in the communication network 120, convert or enforce protocols, establish and enforce quality of service (QoS) for the communication devices, provide applications or services in the communication network 120, translate signals, and/or perform other desired functions to facilitate system interoperability and communication in the communication network 120 (e.g., wireless portion of the communication network 120 or wireline portion of the communication network 120). The communication network 120 further can comprise desired components, such as routers, nodes (e.g., general packet radio service (GPRS) nodes, such as serving GPRS support node (SGSN), gateway GPRS support node (GGSN)), switches, interfaces, controllers, etc., that can facilitate communication of data between communication devices in the communication network environment.

A RAN of the communication network 120 can be associated with (e.g., connected to) the core network (e.g., mobile core network) that can facilitate communications by communication devices (e.g., VAs (e.g., 102, 104, 106, and/or 130), communication devices (e.g., 114, 116, and/or 118), devices (e.g., 124, 126, and/or 128, VAMC 122, IoT device, or other communication device) wirelessly connected to the communication network 120. A communication device (e.g., VA, communication device, device, VAMC, or IoT device) can be communicatively connected to the core network via a base station. The core network can facilitate wireless communication of voice and data associated with communication devices associated with the communication network 120. The core network can facilitate routing voice and data communications between communication devices and/or other communication devices (e.g., phone, computer, VA, email server, multimedia server, audio server, video server, news server, financial or stock information server, other communication devices associated with the IP-based network (e.g., the Internet or an intranet) (not explicitly shown in FIG. 1) of or associated with the communication network 120.

In accordance with various embodiments, the communication network 120 can comprise a macro communication network and/or a micro communication network. The macro communication network can be, can comprise, or can be associated with a core network, a cellular network, an IP-based network, Wi-Fi, gigabit wireless (Gi-Fi) network, Hi-Fi network (e.g., providing higher gigabit data communication than Gi-Fi or Wi-Fi), Bluetooth, ZigBee, etc. The micro communication network can be associated with the macro communication network, wherein the micro communication network typically can operate in a defined local area (e.g., in or in proximity to a home, building, or other defined area). The micro communication network can be, can comprise, or can be associated with Wi-Fi, Gi-Fi, Hi-Fi, Bluetooth, ZigBee, or other communication technology, and/or can be associated with (e.g., connected to) the macro communication network. The micro communication network can be or can comprise, for example a local area network (LAN), that can facilitate connecting certain devices (e.g., communication devices) associated with the micro communication network to each other and/or to the macro communication network.

Respective communication devices (e.g., VA, communication device, device, VAMC, or IoT device) can be associated with (e.g., communicatively connected to) the communication network 120 via a wireless communication connection or a wireline (e.g., wired) communication connection (e.g., via a cell and associated base station). The respective communication devices can operate and communicate in a communication network environment. At various times, a communication device can be communicatively connected via a wireless communication connection(s) to one or more RANs, which can comprise one or more base stations to communicatively connect the communication device to the communication network 120 to enable the communication device to communicate other communication devices associated with (e.g., communicatively connected to) the communication network 120 in the communication network environment. The one or more RANs can comprise, for example, a 3GPP universal mobile telecommunication system (UMTS) terrestrial RAN (UTRAN), an E-UTRAN (e.g., Long Term Evolution (LTE) RAN), 5G or other next generation (xG) RAN, a GSM RAN (GRAN), and/or other type of RAN(s) employing another type of communication technology.

As a communication device(s) (e.g., VA, communication device, device, VAMC, or IoT device) is moved through a wireless communication network environment, at various times, the communication device(s) can be connected (e.g., wirelessly connected) to one of a plurality of base stations or APs (e.g., macro or cellular AP, femto AP, pico AP, wi-fi AP, wi-max AP, hotspot (e.g., hotspot 1.x, hotspot 2.x, where x is an integer number; communication device (e.g., communication device functioning as a mobile hotspot)) that can operate in the wireless communication network environment. An AP (e.g., base station) can serve a specified coverage area to facilitate communication by the communication device(s) or other communication devices in the wireless communication network environment. An AP can serve a respective coverage cell (e.g., macrocell, femtocell, picocell, or other type of cell) that can cover a respective specified area, and the AP can service mobile wireless devices, such as the communication device(s) located in the respective area covered by the respective cell, where such coverage can be achieved via a wireless link (e.g., uplink (UL), downlink (DL)). When an attachment attempt is successful, the communication device(s) can be served by the AP and incoming voice and data traffic can be paged and routed to the communication device(s) through the AP, and outgoing voice and data traffic from the communication device(s) can be paged and routed through the AP to other communication devices in the communication network environment. In an aspect, the communication device(s) can be connected and can communicate wirelessly using virtually any desired wireless technology, including, for example, cellular, Wi-Fi, Gi-Fi, Hi-Fi, Wi-Max, Bluetooth, wireless local area networks (WLAN), or other communication technology.

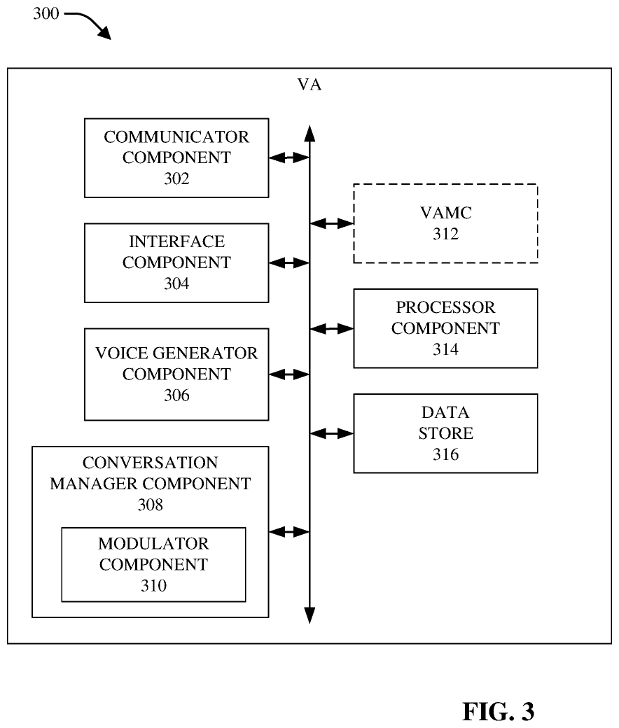

FIG. 3 illustrates a block diagram of an example VA 300, in accordance with various aspects and embodiments of the disclosed subject matter. The VA 300 can comprise, for example, a communicator component 302, an interface component 304, a voice generator component 306, a conversation manager component 308, and a modulator component 310. In some embodiments, the VA 300 can comprise (e.g., optionally can include) a VAMC 312. The VA 300 can be the same as or similar to, and/or can comprise the same or similar functionality as, any of the VAs, as described herein (and vice versa). The VA 300 also can comprise a processor component 314 and data store 316.

The communicator component 302 can transmit information from the VA 300 to a user(s), or a component(s) or device(s) (e.g., another VA, a communication device, a network component or device, or other component or device) and/or can receive information from the user, other component(s), or device(s), such as described herein.

The interface component 304 can comprise one or more interfaces, such as, for example, a display screen (e.g., touch display screen), an audio interface (e.g., microphone(s), speaker(s)), keyboard, keypad, controls, buttons, and/or other interface, that can be used to present information to a user associated with the VA 300 or receive information from the user, or a component(s) or device(s) (e.g., another VA, a communication device, a network component or device, or other component or device), such as information that is input or communicated to the VA 300 by the user or the component(s) or the device(s). The VA 300 can interact with and have a conversation with the user by using the speaker(s) of the interface component 304 to present verbal words to the user, and the VA 300 can receive, via a microphone(s) of the interface component 304, verbal words spoken by the user. As another example, the user can view information (e.g., information relating to the interaction or event) displayed on the display screen of the interface component 304.

The voice generator component 306 can generate one or more voices of the VA 300 for use in communicating (e.g., speaking) verbal words and sounds that can be emitted from the VA 300 via the interface component 304 and/or communicator component 302. A voice generated by the voice generator component 306 can be a virtual or emulated voice that can emulate, mimic, recreate, or sound similar to the actual voice of a human being. The voice can have various characteristics (e.g., word speed, speech cadence, inflection, tone, language, dialect, vocabulary level, or other characteristic) that can define or structure the voice and the speaking (e.g., virtual or emulated speaking) of verbal words by the voice generator component 306.

The conversation manager component 308 can manage (e.g., control, modify, or adjust) the voice (e.g., the characteristics of the virtual or emulated voice) and the emission (e.g., speaking) of verbal words by the voice generator component 306 to facilitate managing a conversation with a user (or another VA or device) based at least in part on sentiment and personality attributes of the user, the context of the user or context of the interaction with the user (or other VA or device), including the verbal words spoken, and the characteristics of the verbal words spoken, by the user (or other VA or device) during the conversation, the environmental conditions associated with the user, and/or the VA personality attributes of the VA 300. The conversation manager component 308 also can determine and manage the verbal words to be emitted by the voice generator component 306 during the conversation, based at least in part on the sentiment and personality attributes of the user, the context of the user or the interaction, including what was said to the VA 300 by the user (or other VA or device) participating in the conversation, the environmental conditions associated with the user, and/or the VA personality attributes of the VA 300. For example, based at least in part on the sentiment and personality attributes of the user, the context, the environmental conditions, and/or the VA personality attributes, the conversation manager component 308 (e.g., in coordination with, or as managed by, the VAMC) can determine a question to ask or a statement to make to the user (or other VA or device) next in a conversation, a response to a question or statement made by the user or other VA to the VA 300, or other conversation to have with the user (or other VA or device). The conversation manager component 308 can coordinate with, be managed by, and/or operate in conjunction with the VAMC to facilitate managing the voice, the determination of verbal words to be emitted, the emission of verbal words, and the overall conversing by the voice generator component 306, based at least in part on the sentiment and personality attributes of the user, the context, the environmental conditions, and/or the VA personality attributes, in accordance with the defined VA management criteria.

The conversation manager component 308 can comprise a modulator component 310 that can be utilized to modulate or adjust the voice, including adjusting the characteristics of the voice, produced by the voice generator component 306. For example, based at least in part on the sentiment and personality attributes of the user, the context, the environmental conditions, and/or the VA personality attributes, the modulator component 310 can adjust (e.g., increase or decrease) the speed and/or cadence of the verbal words emitted by the voice generator component 306, the inflection and/or tone of the voice and/or verbal words emitted by the voice generator component 306, the language and/or dialect of the verbal words emitted by the voice generator component 306, the vocabulary level of the verbal words emitted by the voice generator component 306, the syntax of the conversation, and/or one or more other characteristics of the voice or verbal words to facilitate producing verbal words that can enhance the flow of the conversation and enhance the productivity and results of the conversation and interaction with the user (or VA or other device).

In some embodiments, the VA 300 can comprise (e.g., optionally can comprise) the VAMC 312 that can control the VA 300 during an interaction between the VA 300 and a user, another VA, or another device or component, as more fully described herein. In other embodiments, the VAMC can be separate from, but associated with (e.g., communicatively connected to), the VA 300 to control the VA 300 during an interaction between the VA 300 and a user, another VA, or another device or component, such as more fully described herein. In certain embodiments, even if the VA 300 does not include all of the components of the VAMC, the VA 300 can comprise certain desired components (e.g., AI component, action component, recommendation component, notification component, side effect determination component, cost component, authenticator component, privacy management component, and/or another component) and associated functionality of the VAMC.

The processor component 314 can work in conjunction with the other components (e.g., communicator component 302, interface component 304, voice generator component 306, conversation manager component 308, modulator component 310, VAMC 312, and data store 316) to facilitate performing the various functions of the VA 300. The processor component 314 can employ one or more processors, microprocessors, or controllers that can process data, such as information relating to users, medical care providers, VAs, communication devices or other devices, interactions, events, contexts of users or interactions, status or progress of interactions, medicine, medical care, medical treatment, medical procedures, drugs, foods, products, proposed actions relating to medical care associated with users, costs associated with medical care, health insurance or other insurance, demographic data, data privacy, authorizations to authorize sharing or releasing secured information (e.g., medical, personal, or other sensitive data associated with users, such as patients), access rights to access data, activities relating to interactions, environmental conditions associated with users or interactions, conversations associated with participants of interactions, identifiers or authentication credentials associated with entities, devices, or components, voice generation of the VA 300, characteristics or modulations of one or more voices generated by the VA 300, updates to user profiles of users, parameters, traffic flows, policies, defined VA management criteria, algorithms (e.g., VA management algorithm(s), AI-based algorithms, or other algorithms), protocols, interfaces, tools, and/or other information, to facilitate operation of the VA 300, as more fully disclosed herein, and control data flow between the VA 300 and other components (e.g., other VAs, communication devices, base stations, network devices of the communication network, data sources, applications, or other component or device) associated with the VA 300.

The data store 316 can store data structures (e.g., user data, metadata), code structure(s) (e.g., modules, objects, hashes, classes, procedures) or instructions, information relating to users, medical care providers, VAs, communication devices or other devices, interactions, events, contexts of users or interactions, status or progress of interactions, medicine, medical care, medical treatment, medical procedures, drugs, foods, products, proposed actions relating to medical care associated with users, costs associated with medical care, health insurance or other insurance, demographic data, data privacy, authorizations to authorize sharing or releasing secured information (e.g., medical, personal, or other sensitive data associated with users, such as patients), access rights to access data, activities relating to interactions, environmental conditions associated with users or interactions, conversations associated with participants of interactions, identifiers or authentication credentials associated with entities, devices, or components, voice generation of the VA 300, characteristics or modulations of one or more voices generated by the VA 300, updates to user profiles of users, parameters, traffic flows, policies, defined VA management criteria, algorithms (e.g., VA management algorithm(s), AI-based algorithms, or other algorithms), protocols, interfaces, tools, and/or other information, to facilitate controlling operations associated with the VA 300. In an aspect, the processor component 314 can be functionally coupled (e.g., through a memory bus) to the data store 316 in order to store and retrieve information desired to operate and/or confer functionality, at least in part, to the communicator component 302, interface component 304, voice generator component 306, conversation manager component 308, modulator component 310, VAMC 312, processor component 314, data store 316, or other component, and/or substantially any other operational aspects of the VA 300.

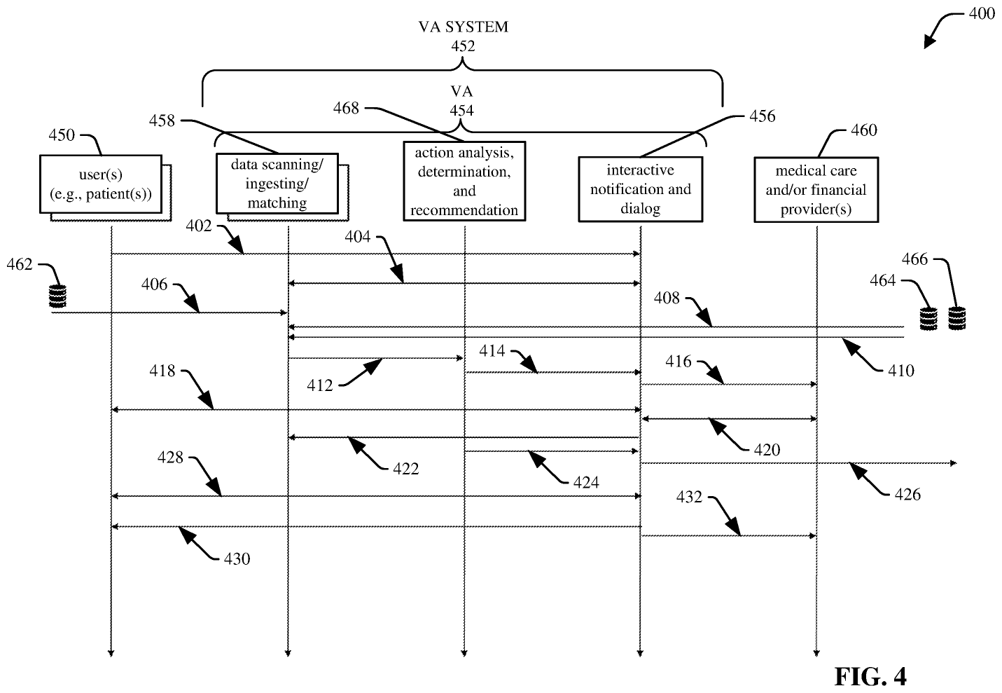

Referring to FIG. 4 (along with FIGS. 1 and 2), FIG. 4 depicts a block diagram of an example VA system process 400 that can manage collection of information associated with users, interactions with users (or other VAs or devices), analysis of information, determinations based on the analysis, proposed actions that can be taken or performed, and presentations of information to users (or other VAs or devices), in accordance with various aspects and embodiments of the disclosed subject matter.

As indicated at reference numeral 402 of the VA system process 400, a user 450 (e.g., patient) can enable a VA system 452 (e.g., system comprising the VAMC), comprising a VA 454, and can select desired automation options that can automate various tasks (e.g., medical or health-related tasks), which can be performed (e.g., automatically performed by the VA system 452, comprising the VA 454, or another VA) on behalf of the user 450. The various tasks can comprise the medical or health-related tasks, or other types of tasks, such as described herein. In some embodiments, the user 450 can opt-in (e.g., optionally authorize) to have the VA system 452 perform the various tasks on behalf of the user 450, wherein, by default, other types of tasks may not be automatically performed by the VA system 452 on behalf of the user 450, if the user 450 has not opted in to have the VA system 452 do so.

The VA system 452 can determine a desirable (e.g., suitable, enhanced, or optimal) place(s) (e.g., location, logically or physically) for the VA system 452, including the VA 454, to operate to be in desired proximity to (e.g., to be adjacent to) the EMRs associated with the user 450 and to respective the privacy concerns of the user 450 and the medical environment as well as enable compliance of the VA system 452 with any applicable privacy laws relating to the information associate with the user 450 or medical environment, based at least in part on the results of analyzing information relating to the user 450, the EMRs associated with the user 450, information relating to user or data privacy laws or regulations, and/or other desired information. The VA system 452 also can determine a desired place(s) for the VA system 452, including the VA 454, to operate in accordance with the desires (e.g., wants, needs, or requirements) of the user 450 based at least in part on analyzing preference information associated with and/or received from the user 450, which can be obtained from the user 450, for example, via interactive notification and dialog 456 between the user 450 and the VA 454. The VA system 452 can distribute the VA 452, another VA(s), and/or other VA system components to a desired place(s) based at least in part on (e.g., in accordance with) the results of analyzing the preference information associated with and/or received from the user 450, information relating to the user 450, the EMRs associated with the user 450, information relating to user or data privacy laws or regulations, and/or other desired information.

In certain embodiments, the VA system 452 can be engaged (e.g., optionally can be engaged) for temporary consultation and/or second opinion with regard to a diagnosis (e.g., automated diagnosis or a diagnosis by a human medical care provider) of a medical condition or a medical treatment for the medical condition, for example, when desired by the user 450, wherein the VA system 452 (e.g., VA 454 of the VA system 452) can perform and provide such temporary consultation and/or second opinion, and/or can contact another VA or another entity (e.g., another human medical care provider) to obtain such temporary consultation and/or second opinion.

As indicated at reference numeral 404 of the VA system process 400, the VA system 452 (e.g., VA 454 of the VA system 452, as part of the data scanning, ingesting, or matching 458) can request, discover, ingest, and/or obtain historical information, including medical information, personal information, biometric information, and/or other information from the user 450, one or more medical care or financial providers 460, one or more applications or sensors, and/or another system (e.g., medical records system, personal information system, medical or health-related system associated with one or more medical or health-related applications or devices, financial provider system, or other information system). In connection with requesting, discovering, ingesting, and/or obtaining such information, the VA system 452 can receive (e.g., obtain or retrieve) user records 462 from the user 450, medical care provider records 464, such as EMRs, associated with the user 450 from the one or more medical care providers (e.g., 460), and/or other medical-related information from one or more other entities 466 (e.g., medical journals or digests, medical news, medical applications or devices, or other entities), as indicated at reference numerals 406, 408, and 410 of the VA system process 400. For instance, the VA system 452 (e.g., via the VA 454 and/or via the communication network 120) can receive personal and/or medical information (e.g., historical or current personal and/or medical information) associated with the user 450 from the user 450 or from the one or more medical care or financial providers 460. In certain embodiments, the VA system 452 (e.g., via the VA 454 and/or via the communication network 120) can learn and understand the profile and history of the user 450 by using a questionnaire directed to the user 450.

The VA system 452 (e.g., via the VA 454 and/or via the communication network 120) also can receive personal and/or medical information associated with the user 450 from one or more applications (e.g., personal, medical, or health-related applications) associated with one or more medical devices or sensors, and/or one or more third-party services, that the user has utilized or with which the user is associated. In certain embodiments, the user 450 can opt in and/or an entity associated with an application can opt in to allow the VA system 452 to obtain such personal and/or medical information.

In some embodiments, the personal and/or medical information associated with the user 450 can comprise biometric information (e.g., information relating to facial features, eye features, fingerprint or handprint features, voice features, dental features, bodily features, or other biometric features) associated with the user 450. The VA system 452 (e.g., via the VA 454 and/or via the communication network 120) can obtain such biometric information from the user 450 directly or from the one or more medical service or financial providers 460, one or more applications, and/or another entity.

At one or more desired times, the VA system 452 (e.g., via the VA 454 and/or via the communication network 120) also can receive updates with regard to personal and/or medical information associated with the user 450, and/or updates with regard to sensor (e.g., medical or health sensor) or biometric information associated with the user 450, from the user 450, the one or more medical care or financial providers 460, applications or services associated with the user 450, and/or other entities or associated databases.

As indicated at reference numeral 412 of the VA system process 400, the VA system 452 (e.g., VA 454 or other component(s) of the VA system 452, as part of the action analysis and recommender functions 468) can determine and generate models (e.g., AI-based models), which can be utilized for learning about users (e.g., user 450 and other users, such as patients) with regard to their medical care and actions (e.g., automated actions or tasks) that VAs (e.g., VA 454 and other VAs) can perform on behalf of and/or for the benefit of the users, with comparison to user histories and data trends in the data associated with the users, based at least in part on the results of an analysis (e.g., an AI-based analysis, utilizing AI functions, algorithms, and models). The actions can comprise the types of actions (e.g., presenting notifications, reminders, or recommendations; advocating on behalf of patients; presenting new or alternate medical treatments or second opinions relating to medical care or treatments; scheduling medical visits; presenting costs or estimated costs relating to medical treatments; and/or other desired types of actions) described herein. The models can be current or historical models that the VA system 452 can learn and develop through AI-based techniques, functions, and algorithms (e.g., through trained Markov chains, trained neural networks, or other type of AI-based model generation and training). The VA 454 can utilize the models to determine action strategies (e.g., strategies relating to determining tasks to perform, performing tasks, presenting recommendations or notifications, or other types of actions, such as described herein) with regard to individual user records (e.g., EMR records and other medical or personal data relating to user 450) or multiple user records.

As part of the analysis of the information (e.g., EMRs, data obtained from sensors or medical devices, or other types of information) associated with one or more users, the VA system 452 can detect a bias or potential bias, if any, of the data inputs and potential demographic diversities (e.g., height, weight, race, or other demographic characteristics versus other recommendation classes). Also, as part of the analysis, the VA system 452 can determine (e.g., calculate) a bias likelihood that can be attached to decisions, determinations insights, actions, or recommendations relating to medical care associated with users (e.g., made by the VA system 452) shared with users (e.g., patients, such as user 450) or medical care providers. The VA system 452 also can integrate different learning techniques, methods, and algorithms develop and determine desirable (e.g., enhanced, useful, or optimal) models relating to medical care associated with users, where the models can be learned based on aggregate but sensitive features associated with users, to share or facilitate sharing such models safely and securely with users (e.g., 450) or medical care providers (e.g., 460).

As indicated at reference numeral 414 of the VA system process 400, the VA system 452 (e.g., VA 454 or other component(s) of the VA system 452, as part of the action analysis and recommender functions 468) can determine proposed actions (e.g., actions or tasks, such as described herein) that the VA 454 can perform on behalf of the user 450 and/or information that the VA 454 can present to the user 450 in connection with the health and medical care of the user 450, and the VA 454 can act and/or advocate on behalf of the user 450 (e.g., patient). For example, based at least in part on the model (e.g., user-specific model) associated with the user 450, information received from the user 450, and/or information obtained from another data source (e.g., e-calendar of the user 450), the VA 454 can determine when to schedule a meeting (e.g., medical visit) or medical testing for the user 450 with a medical care provider (e.g., 460), and, on behalf of the user 450, can contact the medical care provider and schedule the meeting or medical testing for the user 450 with the medical care provider (as indicated at reference numeral 416 of the VA system process 400). As another example, based at least in part on the model or the EMRs associated with the user 450, the VA 454 can recall (e.g., quickly and automatically recall) a specific item(s) of information (e.g., medical or personal information) relating to the user 450, and the VA 454 can present such item(s) of information to the medical care or financial provider (e.g., 460), user 450, or another entity. This can be useful and beneficial as such feature can enhance (e.g., increase or improve) the accuracy of information associated with the user 450 that is presented to the medical care or financial provider (e.g., 460), user 450, or another entity, as the user 450 or medical care or financial provider sometimes may have difficulty recalling specific details (e.g., item(s) of information) associated with the user 450 that are in the EMRs or other information or records associated with the user 450.

As still another example, with regard to a clinical request (e.g., request for medical testing, medical treatment, medical procedure, or other type of clinical request) made to the user 450, the VA 454 can assist or help the user 450 to understand why the clinical request is being made (e.g., when the user 450 questions why the user needs to have another blood screen, or when the user 450 questions why the user should undergo a particular medical treatment), based at least in part on the model associated with the user 450 and/or input received from the medical care provider. This feature can be useful and beneficial as it can remove or reduce the burden and time on the medical care provider (e.g., physician) to explain to the user 450 what is happening and why the clinical request is being made and also can ease or reduce anxiety or concern that the user 450 may have relating to the clinical request.

As yet another example, based at least in part on the model associated with the user 450, the VA 454 can determine whether the user 450 is in sufficiently healthy condition to participate in a particular activity or engage in a particular behavior (e.g., determine whether the user is healthy enough to scuba dive, or play football, or engage in another type of physical activity). Additionally or alternatively, the VA 454 can contact the medical care provider (e.g., 460) on behalf of the user 450 and can ask the medical care provider whether the user 450 is in sufficiently healthy condition to participate in the particular activity or engage in the particular behavior.

As indicated at reference numerals 418, 420, 422, 424, and 426 of the VA system process 400, the VA 454 can interact with the user 450 (e.g., patient) and/or medical care provider(s) for recommendation review and decisions relating to medical care associated with the user 450. For instance, as indicated at reference numeral 418, in accordance with (e.g., adhering to) applicable laws or regulations (e.g., HIPAA or other applicable law or regulation), the VA 454 can interact with the user 450 and can present (e.g., automatically present) certain recommendations, reminders, or notifications to the user 450 (e.g., certain exercise recommendations regarding exercises the user 450 can perform, recommendation or reminder for user 450 to take medication, or other type of recommendation, reminder, or notification), wherein such certain recommendations, reminders, or notifications can be ones that the VA system 452 has determined that the VA 454 can present to the user 450 without having to obtain prior review or approval from the medical care provider (e.g., 460), or can be ones for which the VA 454 already has obtained prior review or approval from the medical care provider. To facilitate adhering to applicable laws or regulations, the VA 454 can determine what information (e.g., what information relating to recommendations, reminders, or notifications) the VA 454 can present to the user 450, what form (e.g., audio, visual, and/or haptic form) the information is to be in, what interface(s) the VA 454 is to utilize to present such information to the user 450, and/or what device(s) (e.g., VA device and/or communication device) the VA 454 is to utilize to present such information to the user 450, such as described herein.

In some embodiments, in connection with determining and presenting information (e.g., proposed actions, recommendations, reminders, or notifications) to the user 450, as indicated at reference numeral 420, the VA 454 can prompt or contact the medical care provider or a group of medical care providers (e.g., the patient's clinician or a panel of clinicians, such as external medical experts, as indicated at reference numeral 426) to have the medical care provider(s) and/or VA system 452 determine a desirable course of action to take with regard to the health or medical care of the user 450 and/or what information to present (e.g., by the VA 454) to the user 450. This can be done with regard to more complex decisions relating to the medical care of the user 450, for example. In certain embodiments, the VA 454 can receive and respond to queries from the medical care provider(s) with regard to information in the health or medical history of the user 450 or medical information pertinent to the medical care of the user 450 that can enable the medical care provider(s) to determine whether, or confirm that, the user 450 fits within a certain class of patients who can be suitable for the course of action (e.g., medical care, treatment, procedure) under consideration. In some instances, the VA 454 can receive, obtain, or ingest additional information relating to the user 450 and/or relating to medical care generally to facilitate performing updated analysis (e.g., AI-based analysis, as part of the action analysis and recommender functions 468) of the newly received information and previous information and updating the model relating to medical care associated with the user 450, responding to queries from the medical care provider(s), and/or determining or recommending a proposed course of action to take with regard to the medical care of the user 450 (as indicated at reference numerals 422 and 424). Upon determination and approval of the desirable course of action, the user 450 can enter into the discussion with the VA 454 and/or the medical care provider(s) to approve (or disapprove) the course of action, remove a portion of the course of action, or request an alteration (e.g., modification or adjustment) to the course of action to facilitate determining an approved course of action that can be agreeable to the user 450, medical care provider(s), and/or VA 454.

In some embodiments, based at least in part on the model associated with the user 450, the VA system 452 can learn or determine (e.g., automatically learn or determine) drug-drug side effects relating to drugs being prescribed, recommended, or considered to or for the user 450, and/or drug-food side effects between a drug(s) and food(s), and the VA 454 can present a notification(s) (e.g., warning(s)) to the user 450 and/or the medical care provider(s) (e.g., before it is too late and the user 450 takes the drugs and/or ingests the food), such as described herein.

As indicated at reference numeral 428 of the VA system process 400, the VA 454 can engage in interactive communication of information (e.g., question and answer chat) with the user 450 with regard to medical care of the user 450 (e.g., recommendation regarding a course of medical care or treatment, explanation regarding the medical care or treatment, explanation regarding costs of such medical care or treatment, reminders or notifications relating to medical care, or other communication of information). For instance, the VA 454 can present, to the user 450, desirable information that can explain to the user 450, and enable the user 450 to better understand, a medical diagnosis relating to a medical condition of the user 450, a cost or estimated cost of a medical treatment or procedure that is being recommended to the user 450, a medical code or an insurance code relating to medical care, treatment, or procedure that is being recommended to the user 450.

In certain embodiments, the VA 454 can interact and communicate with one or more other VAs to determine and/or provide a full medical care planning route, with regard to one or more medical tests, treatments, or procedures the user 450 can undergo during a course of treatment, costs or estimated costs of the course of treatment, and portions there, time table of times of the respective parts of the course of treatment, without the VA 454 or other VA(s) exposing or presenting the queries associated with the VA interactions to the medical care provider(s) (e.g., 460) and while complying with applicable laws and regulations relating to data privacy of patient medical information of the user 450. In some embodiments, to facilitate more regular medical or living care for the user 450 (e.g., for a patient who may desire or need to have regular reminders, recommendations, or decisions with regard to health or medical care), the VA 454 can provide a persistent dialog system to the user 450 that can enable the VA 454 to determine reminders, recommendations, and/or decisions relating to the health and medical care of the user 450 and can present, to the user 450, information relating to such reminders, recommendations, and/or decisions on a regular basis, as desired (e.g., as appropriate for the user 450 and/or in accordance with a preference of the user 450).

As indicated at reference numerals 430 and 432 of the VA system process 400, the VA 454 and one or more other VAs can form a combined hierarchy of VAs that can enable an overall and/or holistic approach to provide overall and/or holistic (e.g., aggregated or mediated) actions, decisions, recommendations, and/or notifications relating to the medical care of the user 450, and the VA 454 and the one or more other VAs can interact and communicate with each other to facilitate determining such actions, decisions, recommendations, and/or notifications. The set of VAs, comprising the VA 454, can be private with regard to the user 450 (e.g., personalized to the user 450 with regard to VA operation, on premises associated with the user 450, or only visible to or accessible by the user 450), or can be shared (e.g., from a practice or larger set of medical care providers).

In some embodiments, the set of VAs can be combined across different medical care specialties such that a first VA can be specialized (e.g., domain specific) to determine actions and provide information relating to cardiac care, a second VA can be specialized to determine actions and provide information relating to dental care, a third VA can be specialized to determine actions and provide information relating to vision care, a fourth VA can be specialized to determine actions and provide information relating to health and physical fitness, and/or another VA can be specialized to determine actions and provide information relating to another type of medical care. Each of the VAs of the set of VAs can provide respective or alternate actions, decisions, or recommendations relating to the health or medical care of the user 450, and/or can negotiate with each other and/or with medical care providers (e.g., physician or medical specialist or expert) to determine overall, finalized, or holistic actions, decisions, or recommendations relating to the medical care of the user 450. One or more of the VAs (e.g., VA 454) can present information relating to the overall, finalized, or holistic actions, decisions, or recommendations to the user 450.

In certain embodiments, respective VAs of the set of VAs can cast respective votes for respective actions, decisions, or recommendations relating to the medical care of the user 450, and a VA (e.g., VA 454) of the set of VAs can tabulate the respective votes and determine an overall or finalized action, decision, or recommendation relating to the medical care of the user 450 based at least in part on the voting results (e.g., the action, decision, or recommendation having the highest vote total relative to other actions, decisions, or recommendations can be selected by the VA 454 as the overall or finalized action, decision, or recommendation). The VA 454 can present, to the user 450, information relating to the overall or finalized action, decision, or recommendation relating to the medical care of the user 450.

In some embodiments, a VA (e.g., VA 454) of the set of VAs can request additional information from another VA of the set of VAs and/or from another entities (e.g., medical care provider or medical expert) that has proposed a different medical treatment for a medical condition than the VA to enable the VA (as well as the other VAs) to better align medical treatment for the medical condition with what other VAs or other entities are recommending for treatment of the medical condition. For instance, the VA 454 or a medical care provider providing medical care to the user 450 with regard to a particular medical condition may not be aware of a newer medical treatment being utilized elsewhere to treat patients with the particular medical condition, wherein one or more other VAs can be recommending such newer medical treatment to treat patients with the particular medical condition. The VA 454 can request information from the one or more other VAs regarding what medical treatments the one or more other VAs are recommending (or medical care providers or medical experts are recommending) to treat patients with the particular medical condition. The VA 454 (or VA system 452) can analyze the information received from the one or more other VAs, and, based at least in part on the results of such analysis (e.g., AI-based analysis and updating of the model), the VA 454 (or VA system 452) can determine that the newer medical treatment is a better medical treatment than the medical treatment the VA 454 would otherwise have recommended, and the VA 454 can present information relating to, and/or recommending, the newer medical treatment to the user 450 and/or medical care provider (e.g., 460) of the user 450.

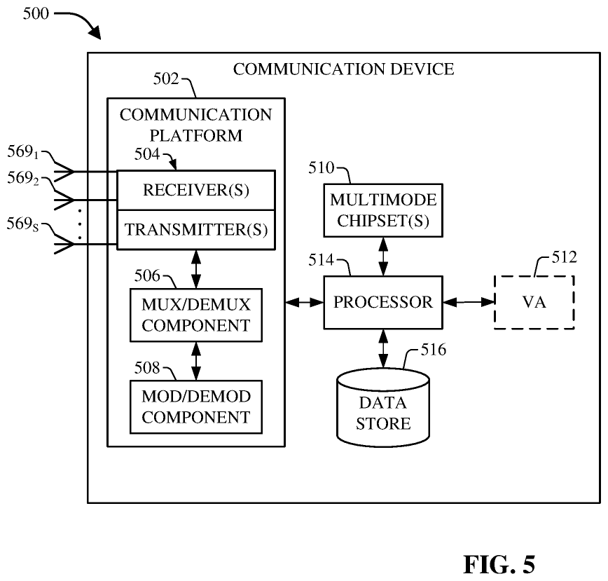

FIG. 5 depicts a block diagram of an example communication device 500 (e.g., communication device) in accordance with various aspects and embodiments of the disclosed subject matter. In accordance with various embodiments, the communication device 500 (e.g., communication device) can be a multimode access terminal, wherein a set of antennas 569₁-569_{S} (wherein S can be a positive integer) can receive and transmit signal(s) from and to wireless devices like access points, access terminals, wireless ports and routers, and so forth, that operate in a radio access network. It should be appreciated that antennas 569₁-569_{S} can be a part of communication platform 502, which comprises electronic components and associated circuitry that provide for processing and manipulation of received signal(s) and signal(s) to be transmitted; e.g., receivers and transmitters 504, multiplexer/demultiplexer (mux/demux) component 506, and modulation/demodulation (mod/demod) component 508.

In some implementations, the communication device 500 can include a multimode operation chipset(s) 510 that can allow the communication device 500 to operate in multiple communication modes in accordance with disparate technical specification for wireless technologies. In an aspect, multimode operation chipset(s) 510 can utilize communication platform 502 in accordance with a specific mode of operation (e.g., voice, global positioning system (GPS), or other mode of operation). In another aspect, multimode operation chipset(s) 510 can be scheduled to operate concurrently (e.g., when S>1) in various modes or within a multitask paradigm.

In certain embodiments, the communication device 500 also can comprise (e.g., optionally can comprise or implement) a VA 512 that can interact with a user, perform services for, and/or perform functions or tasks for or on behalf of, the user, as more fully described herein. The VA 512 can comprise the same or similar functionality as more fully described herein with regard to other systems or methods disclosed herein.

The communication device 500 also can include a processor(s) 514 that can be configured to confer functionality, at least in part, to substantially any electronic component within the communication device 500, in accordance with aspects of the disclosed subject matter. For example, the processor(s) 514 can facilitate enabling the communication device 500 to process data (e.g., symbols, bits, or chips) for multiplexing/demultiplexing, modulation/demodulation, such as implementing direct and inverse fast Fourier transforms, selection of modulation rates, selection of data packet formats, inter-packet times, etc. As another example, the processor(s) 514 can facilitate enabling the communication device 500 to process data relating to messaging, voice calls, or other services (e.g., Internet services or access); information relating to measurements of signal conditions with respect to cells; information relating to cells to facilitate connection to a source cell or target cell; information relating to parameters (e.g., communication device parameters, network-related parameters); information relating to interactions between the communication device 500 and other devices or components (e.g., VA, another communication device), as more fully described herein; and/or other data. In certain embodiments, the processor(s) 514 can process data to facilitate implementing the VA 512 and/or facilitate enabling the VA 512 to interact with a user, perform services for, and/or perform functions or tasks for or on behalf of, the user, as more fully described herein.

The communication device 500 also can contain a data store 516 that can store data structures (e.g., user data, metadata); code structure(s) (e.g., modules, objects, classes, procedures) or instructions; message hashes; neighbor cell list; one or more lists (e.g., whitelist, etc.); information relating to measurements of signal conditions with respect to cells; information relating to cells to facilitate connection to a source cell or target cell; information relating to parameters (e.g., communication device parameters, network-related parameters); information relating to interactions between the communication device 500 and other devices or components (e.g., VA, another communication device); communication device identifier; information relating to voice calls, messaging, or other services associated with the communication device 500; network or device information like policies and specifications; attachment protocols; code sequences for scrambling, spreading and pilot (e.g., reference signal(s)) transmission; frequency offsets; cell IDs; encoding algorithms; compression algorithms; decoding algorithms; decompression algorithms; and so on. In certain embodiments, the data store 516 can store data relating to the VA 512 to facilitate implementing the VA 512 and/or facilitate enabling the VA 512 to interact with a user, perform services for, and/or perform functions or tasks for or on behalf of, the user, as more fully described herein. In an aspect, the processor(s) 514 can be functionally coupled (e.g., through a memory bus) to the data store 516 in order to store and retrieve information (e.g., neighbor cell list; signal quality measurement-related information; cell-related information; parameter information; information relating to messaging, voice calls, or other services (e.g., interactive services); information relating to interactions; frequency offsets; desired algorithms; security code; communication device identifier); and/or VA-related data that can be desired to operate and/or confer functionality, at least in part, to communication platform 502, multimode operation chipset(s) 510, the VA 512, and/or substantially any other operational aspects of the communication device 500.

The aforementioned systems and/or devices have been described with respect to interaction between several components. It should be appreciated that such systems and components can include those components or sub-components specified therein, some of the specified components or sub-components, and/or additional components. Sub-components could also be implemented as components communicatively coupled to other components rather than included within parent components. Further yet, one or more components and/or sub-components may be combined into a single component providing aggregate functionality. The components may also interact with one or more other components not specifically described herein for the sake of brevity, but known by those of skill in the art.

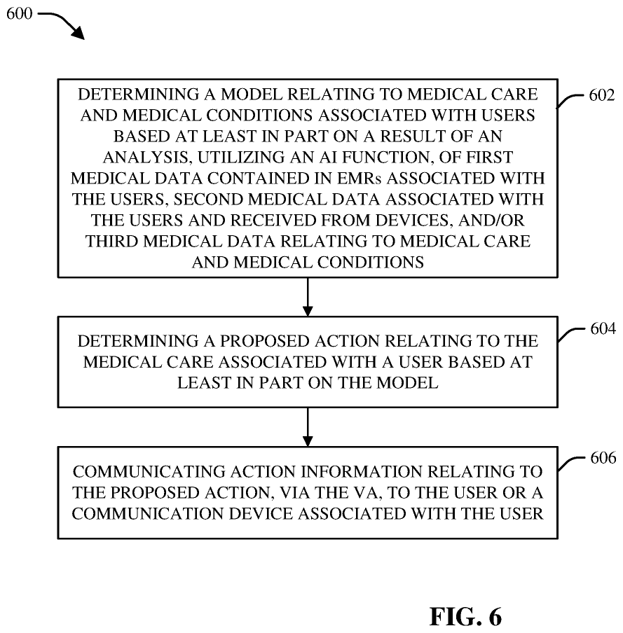

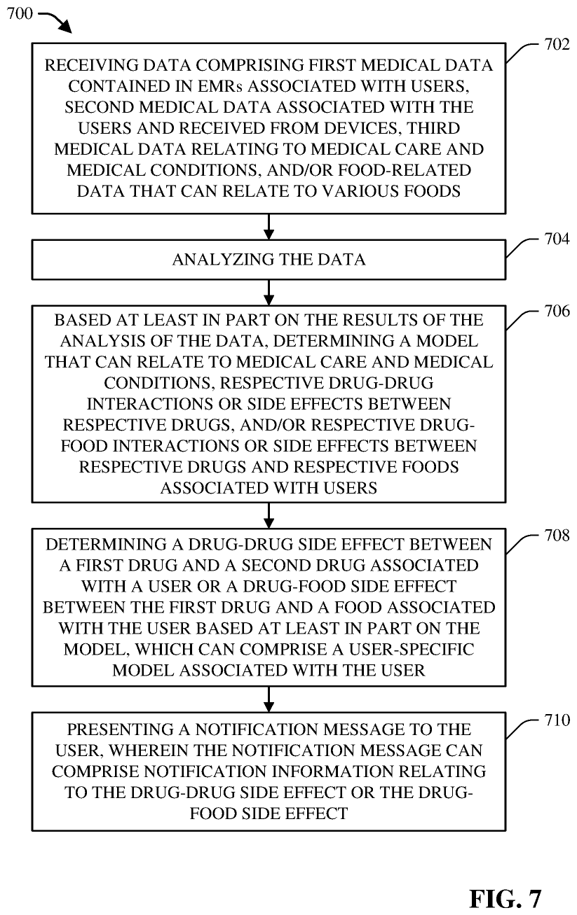

In view of the example systems and/or devices described herein, example methods that can be implemented in accordance with the disclosed subject matter can be further appreciated with reference to flowcharts in FIGS. 6-7. For purposes of simplicity of explanation, example methods disclosed herein are presented and described as a series of acts; however, it is to be understood and appreciated that the disclosed subject matter is not limited by the order of acts, as some acts may occur in different orders and/or concurrently with other acts from that shown and described herein. For example, a method disclosed herein could alternatively be represented as a series of interrelated states or events, such as in a state diagram. Moreover, interaction diagram(s) may represent methods in accordance with the disclosed subject matter when disparate entities enact disparate portions of the methods. Furthermore, not all illustrated acts may be required to implement a method in accordance with the subject specification. It should be further appreciated that the methods disclosed throughout the subject specification are capable of being stored on an article of manufacture to facilitate transporting and transferring such methods to computers for execution by a processor or for storage in a memory.

FIG. 6 illustrates a flow chart of an example method 600 that can manage a VA(s) in interactions with a user (e.g., a patient), including determining, and presenting to the user, information regarding a proposed action relating to medical care of the user, in accordance with various aspects and embodiments of the disclosed subject matter. The method 600 can be employed by, for example, a system comprising the VAMC and/or the VA, a processor component (e.g., of or associated with the VAMC and/or the VA), and/or a data store (e.g., of or associated with the VAMC and/or the VA).

At 602, a model relating to medical care and medical conditions associated with users can be determined based at least in part on a result of an analysis, utilizing an AI function, of first medical data contained in EMRs associated with the users, second medical data associated with the users and received from devices, and/or third medical data relating to medical care and medical conditions. The VAMC or VA can analyze the first medical data, second medical data, and third medical data. The second medical data can comprise, for example, data obtained from health or medical applications, devices, and/or sensors associated with users. In some embodiments, the analysis performed by the VAMC or VA can comprise an AI-based analysis, which can utilize an AI-based technique, function, or algorithm, such as described herein. Based at least in part on the results of the analysis of such data, the VAMC or VA can determine the model relating to the medical care and medical conditions associated with the users, wherein the model can comprise a user-specific model that can pertain to a user.

At 604, a proposed action relating to the medical care associated with a user can be determined based at least in part on the model. At 606, action information relating to the proposed action can be communicated, via the VA, to the user or a communication device associated with the user. Based at least in part on the model (e.g., the user-specific model), the VAMC or VA can determine the proposed action relating to the medical care associated with the user. The proposed action can comprise, one or more determinations, decisions, recommendations, notifications, or other actions that can relate to the health or medical care of the user, such as described herein. The VA can present or communicate the action information relating to the proposed action to the user or a communication device associated with the user, wherein the user can consider, accept, or act on the action information.

FIG. 7 presents a flow chart of an example method 700 that can determine drug-drug interactions or side effects between drugs, and/or drug-food interactions or side effects between a drug and a food, in accordance with various aspects and embodiments of the disclosed subject matter. The method 700 can be employed by, for example, a system comprising a system comprising the VAMC and/or the VA, a processor component (e.g., of or associated with the VAMC and/or the VA), and/or a data store (e.g., of or associated with the VAMC and/or the VA).

At 702, data can be received, wherein the data can comprise first medical data contained in EMRs associated with users, second medical data associated with the users and received from devices, third medical data relating to medical care and medical conditions, and/or food-related data that can relate to various foods. The VAMC or VA can receive the data from respective data sources. For instance, the first medical data can be obtained from one or more databases associated with one or more medical care providers or associated entities (e.g., an entity that stores or archives EMRs associated with users in databases on behalf of medical care providers and users (e.g., patients)).

At 704, the data can be analyzed. The VAMC or VA can analyze the first medical data, second medical data, third medical data, and/or the food-related data. In some embodiments, as part of the analysis, the VAMC or VA can utilize AI-based techniques, functions, and/or algorithms to analyze the data and learn from the data (e.g., learn respective data patterns in the data, learn interactions between drugs, learns interactions between drugs and foods, learn about medical care, medical conditions, medical treatments, and medical procedures, and/or learn other characteristics and features associated with the data), such as described herein. In some embodiments, the first medical data or the second medical data can comprise first drug data relating to a first drug and a second drug, and the third medical data can comprise second drug data relating to ingredients of the first drug and the second drug. The food-related data can comprise respective food-related information related to respective foods, including respective ingredients of the respective foods.

At 706, based at least in part on the results of the analysis of the data, a model can be determined, wherein the model can relate to medical care and medical conditions, respective drug-drug interactions or side effects between respective drugs, and/or respective drug-food interactions or side effects between respective drugs and respective foods associated with users. For instance, based at least in part on the analysis results, the VAMC or VA can determine the model, such as described herein. In some embodiments, the model can comprise respective user-specific models that can be associated with respective users.

At 708, a drug-drug side effect between a first drug and a second drug associated with a user or a drug-food side effect between the first drug and a food associated with the user can be determined based at least in part on the model, which can comprise a user-specific model associated with the user. The VAMC or VA can analyze the model, including what was learned and modeled with regard to respective drug-drug interactions between respective drugs or what was learned and modeled with regard to respective drug-food interactions between respective drugs and respective foods. Based at least in part on the results of analyzing the model, the VAMC or VA can determine the drug-drug side effect between the first drug and the second drug associated with the user or the drug-food side effect between the first drug and the food associated with the user.

At 710, a notification message can be presented to the user, wherein the notification message can comprise notification information relating to the drug-drug side effect or the drug-food side effect. In some embodiments, the VA can present (e.g., via a display screen, audio speaker, or other interface of the VA) the notification message to the user to facilitate notifying or warning the user of the drug-drug side effect or the drug-food side effect. In other embodiments, the VA can present (e.g., communicate) the notification message to a communication device associated with the user, wherein the communication device can present (e.g., via a display screen, audio speaker, or other interface of the communication device) the notification message to the user to facilitate notifying or warning the user of the drug-drug side effect or the drug-food side effect.

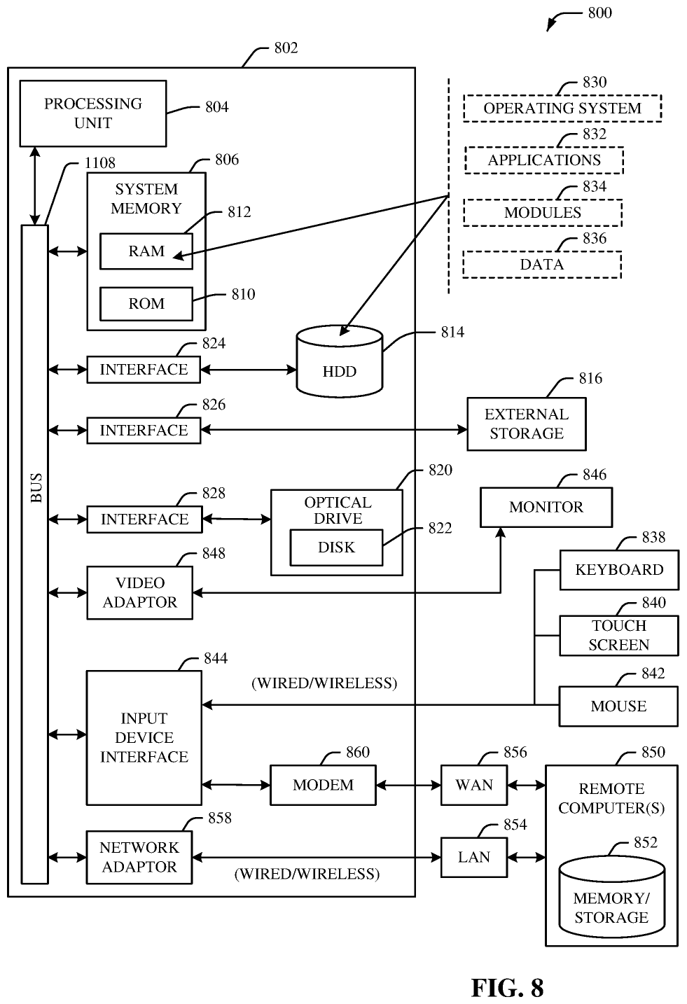

In order to provide additional context for various embodiments described herein, FIG. 8 and the following discussion are intended to provide a brief, general description of a suitable computing environment 800 in which the various embodiments of the embodiments described herein can be implemented. While the embodiments have been described above in the general context of computer-executable instructions that can run on one or more computers, those skilled in the art will recognize that the embodiments can be also implemented in combination with other program modules and/or as a combination of hardware and software.

Generally, program modules include routines, programs, components, data structures, etc., that perform particular tasks or implement particular abstract data types. Moreover, those skilled in the art will appreciate that the methods can be practiced with other computer system configurations, including single-processor or multiprocessor computer systems, minicomputers, mainframe computers, Internet of Things (IoT) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, and/or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible and/or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 8, the example environment 800 for implementing various embodiments of the aspects described herein includes a computer 802, the computer 802 including a processing unit 804, a system memory 806 and a system bus 808. The system bus 808 couples system components including, but not limited to, the system memory 806 to the processing unit 804. The processing unit 804 can be any of various commercially available processors. Dual microprocessors and other multi-processor architectures can also be employed as the processing unit 804.

The system bus 808 can be any of several types of bus structure that can further interconnect to a memory bus (with or without a memory controller), a peripheral bus, and a local bus using any of a variety of commercially available bus architectures. The system memory 806 includes ROM 810 and RAM 812. A basic input/output system (BIOS) can be stored in a non-volatile memory such as ROM, erasable programmable read only memory (EPROM), EEPROM, which BIOS contains the basic routines that help to transfer information between elements within the computer 802, such as during startup. The RAM 812 can also include a high-speed RAM such as static RAM for caching data.

The computer 802 further includes an internal hard disk drive (HDD) 814 (e.g., EIDE, SATA), one or more external storage devices 816 (e.g., a magnetic floppy disk drive (FDD) 816, a memory stick or flash drive reader, a memory card reader, etc.) and an optical disk drive 820 (e.g., which can read or write from a CD-ROM disc, a DVD, a BD, etc.). While the internal HDD 814 is illustrated as located within the computer 802, the internal HDD 814 can also be configured for external use in a suitable chassis (not shown). Additionally, while not shown in environment 800, a solid state drive (SSD) could be used in addition to, or in place of, an HDD 814. The HDD 814, external storage device(s) 816 and optical disk drive 820 can be connected to the system bus 808 by an HDD interface 824, an external storage interface 826 and an optical drive interface 828, respectively. The interface 824 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 802, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 812, including an operating system 830, one or more application programs 832, other program modules 834 and program data 836. All or portions of the operating system, applications, modules, and/or data can also be cached in the RAM 812. The systems and methods described herein can be implemented utilizing various commercially available operating systems or combinations of operating systems.

Computer 802 can optionally comprise emulation technologies. For example, a hypervisor (not shown) or other intermediary can emulate a hardware environment for operating system 830, and the emulated hardware can optionally be different from the hardware illustrated in FIG. 8. In such an embodiment, operating system 830 can comprise one virtual machine (VM) of multiple VMs hosted at computer 802. Furthermore, operating system 830 can provide runtime environments, such as the Java runtime environment or the .NET framework, for applications 832. Runtime environments are consistent execution environments that allow applications 832 to run on any operating system that includes the runtime environment. Similarly, operating system 830 can support containers, and applications 832 can be in the form of containers, which are lightweight, standalone, executable packages of software that include, e.g., code, runtime, system tools, system libraries and settings for an application.

Further, computer 802 can be enable with a security module, such as a trusted processing module (TPM). For instance with a TPM, boot components hash next in time boot components, and wait for a match of results to secured values, before loading a next boot component. This process can take place at any layer in the code execution stack of computer 802, e.g., applied at the application execution level or at the operating system (OS) kernel level, thereby enabling security at any level of code execution.

A user can enter commands and information into the computer 802 through one or more wired/wireless input devices, e.g., a keyboard 838, a touch screen 840, and a pointing device, such as a mouse 842. Other input devices (not shown) can include a microphone, an infrared (IR) remote control, a radio frequency (RF) remote control, or other remote control, a joystick, a virtual reality controller and/or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 804 through an input device interface 844 that can be coupled to the system bus 808, but can be connected by other interfaces, such as a parallel port, an IEEE 1394 serial port, a game port, a USB port, an IR interface, a BLUETOOTH® interface, etc.

A monitor 846 or other type of display device can be also connected to the system bus 808 via an interface, such as a video adapter 848. In addition to the monitor 846, a computer typically includes other peripheral output devices (not shown), such as speakers, printers, etc.

The computer 802 can operate in a networked environment using logical connections via wired and/or wireless communications to one or more remote computers, such as a remote computer(s) 850. The remote computer(s) 850 can be a workstation, a server computer, a router, a personal computer, portable computer, microprocessor-based entertainment appliance, a peer device or other common network node, and typically includes many or all of the elements described relative to the computer 802, although, for purposes of brevity, only a memory/storage device 852 is illustrated. The logical connections depicted include wired/wireless connectivity to a local area network (LAN) 854 and/or larger networks, e.g., a wide area network (WAN) 856. Such LAN and WAN networking environments are commonplace in offices and companies, and facilitate enterprise-wide computer networks, such as intranets, all of which can connect to a global communications network, e.g., the Internet.

When used in a LAN networking environment, the computer 802 can be connected to the local network 854 through a wired and/or wireless communication network interface or adapter 858. The adapter 858 can facilitate wired or wireless communication to the LAN 854, which can also include a wireless access point (AP) disposed thereon for communicating with the adapter 858 in a wireless mode.

When used in a WAN networking environment, the computer 802 can include a modem 860 or can be connected to a communications server on the WAN 856 via other means for establishing communications over the WAN 856, such as by way of the Internet. The modem 860, which can be internal or external and a wired or wireless device, can be connected to the system bus 808 via the input device interface 844. In a networked environment, program modules depicted relative to the computer 802 or portions thereof, can be stored in the remote memory/storage device 852. It will be appreciated that the network connections shown are example and other means of establishing a communications link between the computers can be used.

When used in either a LAN or WAN networking environment, the computer 802 can access cloud storage systems or other network-based storage systems in addition to, or in place of, external storage devices 816 as described above. Generally, a connection between the computer 802 and a cloud storage system can be established over a LAN 854 or WAN 856, e.g., by the adapter 858 or modem 860, respectively. Upon connecting the computer 802 to an associated cloud storage system, the external storage interface 826 can, with the aid of the adapter 858 and/or modem 860, manage storage provided by the cloud storage system as it would other types of external storage. For instance, the external storage interface 826 can be configured to provide access to cloud storage sources as if those sources were physically connected to the computer 802.

The computer 802 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

It is to be noted that aspects, features, and/or advantages of the disclosed subject matter can be exploited in substantially any wireless telecommunication or radio technology, e.g., Wi-Fi; Gi-Fi; Hi-Fi; Bluetooth; worldwide interoperability for microwave access (WiMAX); enhanced general packet radio service (enhanced GPRS); third generation partnership project (3GPP) long term evolution (LTE); third generation partnership project 2 (3GPP2) ultra mobile broadband (UMB); 3GPP universal mobile telecommunication system (UMTS); high speed packet access (HSPA); high speed downlink packet access (HSDPA); high speed uplink packet access (HSUPA); GSM (global system for mobile communications) EDGE (enhanced data rates for GSM evolution) radio access network (GERAN); UMTS terrestrial radio access network (UTRAN); LTE advanced (LTE-A); Z-Wave; Zigbee; and other 802.XX wireless technologies and/or legacy telecommunication technologies. Additionally, some or all of the aspects described herein can be exploited in legacy telecommunication technologies, e.g., GSM. In addition, mobile as well non-mobile networks (e.g., the internet, data service network such as internet protocol television (IPTV), etc.) can exploit aspects or features described herein.

Wi-Fi, or Wireless Fidelity, can enable connection to the Internet from a couch at home, in a hotel room, or a conference room at work, without wires. Wi-Fi is a wireless technology similar to that used in a cell phone that can enable such devices, e.g., computers, to send and receive data indoors and out; anywhere within the range of a base station. Wi-Fi networks use radio technologies called IEEE 802.11 (a, b, g, etc.) to provide secure, reliable, fast wireless connectivity. A Wi-Fi network can be used to connect computers to each other, to the Internet, and to wired networks (which use IEEE 802.3 or Ethernet). Wi-Fi networks can operate in the unlicensed 2.4 and 5 GHz radio bands, at an 11 Mbps (802.11a) or 54 Mbps (802.11b) data rate, for example, or with products that contain both bands (dual band), so the networks can provide real-world performance similar to the basic 10BaseT wired Ethernet networks used in many offices.

As used in this disclosure, in some embodiments, the terms “component,” “system,” “interface,” and the like can refer to, or comprise, a computer-related entity or an entity related to an operational apparatus with one or more specific functionalities, wherein the entity can be either hardware, a combination of hardware and software, software, or software in execution, and/or firmware. As an example, a component can be, but is not limited to being, a process running on a processor, a processor, an object, an executable, a thread of execution, computer-executable instructions, a program, and/or a computer. By way of illustration and not limitation, both an application running on a server and the server can be a component.

One or more components can reside within a process and/or thread of execution and a component can be localized on one computer and/or distributed between two or more computers. In addition, these components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software application or firmware application executed by one or more processors, wherein the processor can be internal or external to the apparatus and can execute at least a part of the software or firmware application. As yet another example, a component can be an apparatus that provides specific functionality through electronic components without mechanical parts, the electronic components can comprise a processor therein to execute software or firmware that confer(s) at least in part the functionality of the electronic components. In an aspect, a component can emulate an electronic component via a virtual machine, e.g., within a cloud computing system. While various components have been illustrated as separate components, it will be appreciated that multiple components can be implemented as a single component, or a single component can be implemented as multiple components, without departing from example embodiments.

Various aspects or features described herein can be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable device, machine-readable device, computer-readable carrier, computer-readable media, machine-readable media, computer-readable (or machine-readable) storage/communication media. For example, computer-readable media can comprise, but are not limited to, a magnetic storage device, e.g., hard disk; floppy disk; magnetic strip(s); an optical disk (e.g., compact disk (CD), a digital video disc (DVD), a Blu-ray Disc™ (BD)); a smart card; a flash memory device (e.g., card, stick, key drive); and/or a virtual device that emulates a storage device and/or any of the above computer-readable media. Of course, those skilled in the art will recognize many modifications can be made to this configuration without departing from the scope or spirit of the various embodiments. In accordance with various implementations, computer-readable storage media can be non-transitory computer-readable storage media and/or a computer-readable storage device can comprise computer-readable storage media.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. A processor can be or can comprise, for example, multiple processors that can include distributed processors or parallel processors in a single machine or multiple machines. Additionally, a processor can comprise or refer to an integrated circuit, an application specific integrated circuit (ASIC), a digital signal processor (DSP), a programmable gate array (PGA), a field PGA (FPGA), a programmable logic controller (PLC), a complex programmable logic device (CPLD), a state machine, a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor may also be implemented as a combination of computing processing units.

A processor can facilitate performing various types of operations, for example, by executing computer-executable instructions. When a processor executes instructions to perform operations, this can include the processor performing (e.g., directly performing) the operations and/or the processor indirectly performing operations, for example, by facilitating (e.g., facilitating operation of), directing, controlling, or cooperating with one or more other devices or components to perform the operations. In some implementations, a memory can store computer-executable instructions, and a processor can be communicatively coupled to the memory, wherein the processor can access or retrieve computer-executable instructions from the memory and can facilitate execution of the computer-executable instructions to perform operations.

In certain implementations, a processor can be or can comprise one or more processors that can be utilized in supporting a virtualized computing environment or virtualized processing environment. The virtualized computing environment may support one or more virtual machines representing computers, servers, or other computing devices. In such virtualized virtual machines, components such as processors and storage devices may be virtualized or logically represented.

In the subject specification, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory.

By way of illustration, and not limitation, nonvolatile memory can include read only memory (ROM), programmable ROM (PROM), electrically programmable ROM (EPROM), electrically erasable ROM (EEPROM), or flash memory. Volatile memory can include random access memory (RAM), which acts as external cache memory. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), and direct Rambus RAM (DRRAM). Additionally, the disclosed memory components of systems or methods herein are intended to comprise, without being limited to comprising, these and any other suitable types of memory.

As used in this application, the terms “component”, “system”, “platform”, “framework”, “layer”, “interface”, “agent”, and the like, can refer to and/or can include a computer-related entity or an entity related to an operational machine with one or more specific functionalities. The entities disclosed herein can be either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server can be a component. One or more components may reside within a process and/or thread of execution and a component may be localized on one computer and/or distributed between two or more computers.

In another example, respective components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

Terms like “user equipment” (UE), “mobile station,” “mobile,” “wireless device,” “wireless communication device,” “subscriber station,” “subscriber equipment,” “access terminal,” “terminal,” “handset,” and similar terminology, as used herein, can refer to a wireless device utilized by a subscriber or user of a wireless communication service to receive or convey data, control, voice, video, sound, gaming, or substantially any data-stream or signaling-stream. The foregoing terms are utilized interchangeably in the subject specification and related drawings. Likewise, the terms “access point” (AP), “base station,” “node B,” “evolved node B” (eNode B or eNB), “home node B” (HNB), “home access point” (HAP), and the like are utilized interchangeably in the subject application, and refer to a wireless network component or appliance that serves and receives data, control, voice, video, sound, gaming, or substantially any data-stream or signaling-stream from a set of subscriber stations. Data and signaling streams can be packetized or frame-based flows. Further, the terms “device,” “communication device,” “mobile device,” “entity,” and the like can be employed interchangeably throughout, unless context warrants particular distinctions among the terms.

Furthermore, the terms “user,” “entity,” “subscriber,” “customer,” “consumer,” “owner,” “agent,” and the like can be employed interchangeably throughout the subject specification, unless context warrants particular distinction(s) among the terms. It should be appreciated that such terms can refer to human entities or automated components supported through artificial intelligence (e.g., a capacity to make inference based on complex mathematical formalisms), which can provide simulated vision, sound recognition and so forth.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

As used herein, the terms “example,” “exemplary,” and/or “demonstrative” are utilized to mean serving as an example, instance, or illustration. For the avoidance of doubt, the subject matter disclosed herein is not limited by such examples. In addition, any aspect or design described herein as an “example,” “exemplary,” and/or “demonstrative” is not necessarily to be construed as preferred or advantageous over other aspects or designs, nor is it meant to preclude equivalent exemplary structures and techniques known to those of ordinary skill in the art. Furthermore, to the extent that the terms “includes,” “has,” “contains,” and other similar words are used in either the detailed description or the claims, such terms are intended to be inclusive, in a manner similar to the term “comprising” as an open transition word, without precluding any additional or other elements.

Reference throughout this specification to “one embodiment,” or “an embodiment,” means that a particular feature, structure, or characteristic described in connection with the embodiment is included in at least one embodiment. Thus, the appearances of the phrase “in one embodiment,” “in one aspect,” or “in an embodiment,” in various places throughout this specification are not necessarily all referring to the same embodiment. Furthermore, the particular features, structures, or characteristics can be combined in any suitable manner in one or more embodiments.

The term “facilitate” as used herein is in the context of a system, device or component “facilitating” one or more actions or operations, in respect of the nature of complex computing environments in which multiple components and/or multiple devices can be involved in some computing operations. In this regard, a computing device or component can facilitate an operation by playing any part in accomplishing the operation. When operations of a component are described herein, it is thus to be understood that where the operations are described as facilitated by the component, the operations can be optionally completed with the cooperation of one or more other computing devices or components, such as, but not limited to, a virtual assistant (VA), virtual assistant management component (VAMC), AI component, communication devices, processors, data stores, sensors, antennae, audio and/or visual output devices, or other devices.

It is to be appreciated and understood that components (e.g., communication device, virtual assistant (VA), virtual assistant management component (VAMC), communication network, processor component, data store, or other component or device), as described with regard to a particular system or method, can include the same or similar functionality as respective components (e.g., respectively named components or similarly named components) as described with regard to other systems or methods disclosed herein.

The above description of illustrated embodiments of the subject disclosure, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as those skilled in the relevant art can recognize.

In this regard, while the subject matter has been described herein in connection with various embodiments and corresponding figures, where applicable, it is to be understood that other similar embodiments can be used or modifications and additions can be made to the described embodiments for performing the same, similar, alternative, or substitute function of the disclosed subject matter without deviating therefrom. Therefore, the disclosed subject matter should not be limited to any single embodiment described herein, but rather should be construed in breadth and scope in accordance with the appended claims below.

What has been described above includes examples of systems, methods, and techniques that provide advantages of the disclosed subject matter. It is, of course, not possible to describe every conceivable combination of components or methods for purposes of describing the disclosed subject matter, but one of ordinary skill in the art may recognize that many further combinations and permutations of the disclosed subject matter are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

What is claimed is:

1. A method, comprising:
determining, by a system comprising a processor, a model relating to medical care and medical conditions associated with user identities based on a result of an analysis, utilizing an artificial intelligence function, of first medical data contained in electronic medical records associated with the user identities, second medical data associated with the user identities and received from devices, and third medical data relating to the medical care and the medical conditions;
determining, by the system, a proposed action relating to the medical care associated with a user identity based on the model, wherein the user identities comprise the user identity; and
communicating, via a virtual assistant device of the system, action information relating to the proposed action to a device associated with the user identity, wherein the devices comprise the device.

2. The method of claim 1, further comprising:
determining, by the system, a drug-drug side effect between a first drug and a second drug associated with the user identity or a drug-food side effect between the first drug and a food associated with the user identity based on the model and food information relating to the food, wherein the first medical data or the second medical data comprises first drug information relating to the first drug and the second drug, and wherein the third medical data comprises second drug information relating to ingredients of the first drug and the second drug,
wherein the communicating of the action information relating to the proposed action comprises, communicating, by the virtual assistant device, notification information to the device for presentation to a user associated with the user identity, and wherein the notification information notifies and warns the user of the drug-drug side effect between the first drug and the second drug or the drug-food side effect between the first drug and the food.

3. The method of claim 2, further comprising:
learning, by the system, the drug-drug side effect between the first drug and the second drug associated with the user identity or the drug-food side effect between the first drug and the food associated with the user identity based on analysis, utilizing the artificial intelligence function, of the first drug information, the second drug information, and the food information.

4. The method of claim 1, wherein the electronic medical records are first electronic medical records, and wherein the method further comprises:
receiving, by the virtual assistant device, updated medical data associated with the user identity from a user associated with the user identity, second electronic medical records associated with the user identity, or a sensor or a medical device associated with the user; and
modifying, by the system, the model to generate a user-specific model based on analysis, utilizing the artificial intelligence function, of the model, the updated medical data associated with the user identity, and historical medical data associated with the user identity, wherein the historical medical data comprises a portion of the first medical data, the second medical data, or the third medical data, and wherein the determining of the proposed action relating to the medical care associated with the user identity comprises determining the proposed action relating to the medical care associated with the user identity based on the user-specific model.

5. The method of claim 4, wherein the virtual assistant device is a first virtual assistant device, wherein the portion is a first portion, wherein the receiving of the updated medical data comprises: prior to, and within a defined amount of time of, a medical care visit with a medical care provider by the user, receiving, by the virtual assistant device, a second portion of the updated medical data from the user, the sensor, or the medical device, and wherein the method further comprises:
prior to the medical care visit with the medical care provider by the user, presenting, via a first interface associated with the first virtual assistant device, the second portion of the updated medical data to the medical care provider in connection with the medical care visit; or
prior to the medical care visit with the medical care provider by the user:
communicating, by the first virtual assistant device, the second portion of the updated medical data to a second virtual assistant device associated with the medical care provider, and
presenting, via a second interface associated with the second virtual assistant device, the second portion of the updated medical data to the medical care provider in connection with the medical care visit.

6. The method of claim 4, wherein the virtual assistant device is a first virtual assistant device, wherein the portion is a first portion, wherein the medical care provider is a first medical care provider, and wherein the receiving of the updated medical data comprises:
receiving, by the first virtual assistant device, a second portion of the electronic medical records from a first device or a second virtual assistant device associated with a second medical care provider; and
receiving, by the first virtual assistant device, a third portion of the electronic medical records from a second device or a third virtual assistant device associated with a third medical care provider.

7. The method of claim 1, wherein the virtual assistant device is a first virtual assistant device, wherein the device is a first device, and wherein the method further comprises:
perceiving, by the first virtual assistant device, a medical condition associated with a user associated with the user identity while the user is at a first location;
based on the perceiving, generating, by the first virtual assistant device, medical condition information relating to the medical condition; and
communicating, by the first virtual assistant device, the medical condition information to a second virtual assistant device or a second device associated with a medical care provider identity, wherein the second virtual assistant device or the second device is located at a second location.

8. The method of claim 1, further comprising:
determining, by the system, a cost or an estimated cost of a medical treatment regimen associated with the user identity based on the model; and
presenting, via an interface associated with the virtual assistant device, cost information relating to the cost or the estimated cost to a user associated with the user identity, wherein the determining of the proposed action comprises determining the proposed action relating to the medical care based on the cost information or based on feedback information relating to the medical treatment regimen, and wherein the feedback information is received by the virtual assistant device from the user.

9. The method of claim 1, wherein the virtual assistant device is a first virtual assistant device, and wherein the method further comprises:
communicating, by the first virtual assistant device, a request for information relating to a medical condition associated with the user identity to a second virtual assistant device based on determining that the second virtual assistant device has access to medical field information relating to a medical field associated with the medical condition; and
receiving, by the first virtual assistant device, the information relating to the medical condition from the second virtual assistant device, wherein the determining of the proposed action comprises determining the proposed action relating to the medical care based on the model and analysis of the information relating to the medical condition.

10. The method of claim 9, wherein the first virtual assistant device accesses first medical treatment information relating to a first type of medical treatment to utilize to treat the medical condition, wherein the information relating to the medical condition comprises second medical treatment information relating to a second type of medical treatment to utilize to treat the medical condition, wherein the determining of the proposed action comprises determining which of the first type of medical treatment, the second type of medical treatment, or a third type of medical treatment is to be utilized or recommended to treat the medical condition based on the model and analysis of the first medical treatment information and the second medical treatment information, and wherein the third type of medical treatment is determined based on the first type of medical treatment and the second type of medical treatment.

11. The method of claim 1, further comprising:
receiving, by the virtual assistant device from a medical care provider, a request for information relating to a medical or health history associated with the user identity;
identifying, by the virtual assistant device, the information relating to the medical or health history associated with the user identity in the model, a portion of the electronic medical records that is determined to be associated with the user identity, or sensor data associated with the user identity;
presenting, via an interface of the virtual assistant device to the medical care provider, the information relating to the medical or health history associated with the user identity.

12. The method of claim 1, further comprising:
determining, by the virtual assistant device, a medication a user associated with the user identity is scheduled to take, or an exercise the user is to perform, based on the model; and
presenting, via an interface of the virtual assistant device, to the user, a reminder message or a recommendation message to take the medication or perform the exercise.

13. A system, comprising:
a processor; and
a memory that stores executable instructions that, when executed by the processor, facilitate performance of operations, comprising:
determining a model relating to medical care and medical conditions associated with user identities based on a result of an analysis, utilizing an artificial intelligence function, of first medical information contained in electronic medical records associated with the user identities, second medical information associated with the user identities and received from devices associated with the user identities, and third medical information relating to the medical care and the medical conditions;
determining a proposed action relating to the medical care associated with a user identity based on the model, wherein the user identities comprise the user identity; and
presenting, via a virtual assistant device, action information, relating to the proposed action, to a device associated with the user identity, wherein the devices comprise the device.

14. The system of claim 13, wherein the operations further comprise:
determining a drug-to-drug side effect between a first drug and a second drug associated with the user identity or a drug-to-food side effect between the first drug and a food associated with the user identity based on the model and food information relating to the food, wherein the first medical information or the second medical information comprises first drug information relating to the first drug or the second drug, wherein the third medical information comprises second drug information relating to ingredients of the first drug or the second drug, wherein the presenting of the action information relating to the proposed action comprises presenting, by the virtual assistant device, notification information to the device for presentation to a user associated with the user identity, and wherein the notification information notifies and warns the user of the drug-to-drug side effect between the first drug and the second drug or the drug-to-food side effect between the first drug and the food.

15. The system of claim 13, wherein the operations further comprise:
determining, by the virtual assistant device, that first information relating to a medical condition associated with the user identity is subject to a higher privacy level based on a defined privacy criterion;
determining, by the virtual assistant device, that second information relating to the user identity is subject to a lower privacy level based on the defined privacy criterion;
in response to determining that the first information is subject to the higher privacy level, presenting, via a first interface of the virtual assistant device, the first information to the user, wherein the first interface is determined to be able to provide privacy of the first information at the higher privacy level for the presenting of the first information to the user, and wherein the first information presented via the first interface is only able to be perceived by the user; and
in response to determining that the second information is subject to the lower privacy level, presenting, via a second interface of the virtual assistant device, the second information to the user, wherein the second interface is determined to be able to provide privacy of the second information at the lower privacy level for the presenting of the second information to the user, and wherein the second information presented via the second interface is able to be perceived by the user and another user other than the user.

16. The system of claim 13, wherein the virtual assistant device is a first virtual assistant device, wherein the device is a first device, and wherein the operations further comprise:
determining that a user associated with the user identity has a medical condition based on a user-specific model associated with the user, wherein the user-specific model is determined based on the model and a portion of the first medical information, the second medical information, or the third medical information that pertains to the user;
receiving, by the first virtual assistant device from a second virtual assistant device, medical treatment information relating to a medical treatment for the medical condition, wherein the second virtual assistant device is not associated with a medical care provider that has provided medical care to the user;
determining that the medical treatment has not been considered, by the medical care provider or the user, to treat the user for the medical condition based on analysis of the model, the user-specific model, or the portion of the first medical information, the second medical information, or the third medical information that pertains to the user;
in response to determining that the medical treatment has not been considered by the medical care provider or the user, presenting, by the first virtual assistant device, the medical treatment information relating to the medical treatment for the medical condition to the user or to the medical care provider via a third virtual assistant device or a second device associated with the medical care provider.

17. The system of claim 13, wherein the operations further comprise:
determining a cost or an estimated cost of a medical treatment associated with the user identity based on the model; and
presenting, via an interface associated with the virtual assistant device, cost information relating to the cost or the estimated cost to a user associated with the user identity, wherein the determining of the proposed action comprises determining the proposed action relating to the medical care based on the cost information or based on feedback information relating to the medical treatment, and wherein the feedback information is received by the virtual assistant device from the user.

18. The system of claim 13, wherein the operations further comprise:
determining a medication a user associated with the user identity is scheduled to take, or an exercise the user is to perform, based on the model; and
presenting, via an interface of the virtual assistant device, to the user, a reminder message or a recommendation message to take the medication or perform the exercise.

19. A non-transitory machine-readable medium, comprising executable instructions that, when executed by a processor, facilitate performance of operations, comprising:
determining a model relating to medical care and medical conditions associated with user identities based on a result of an analysis, utilizing an artificial intelligence function, of first medical data contained in electronic medical records associated with the user identities, second medical data associated with the user identities and received from devices associated with the user identities, and third medical data relating to the medical care and the medical conditions;
determining a proposed action relating to the medical care associated with a user identity based on the model, wherein the user identities comprise the user identity; and
presenting, via a virtual assistant function, action information, relating to the proposed action, to a user associated with the user identity.

20. The non-transitory machine-readable medium of claim 19, wherein the electronic medical records are first electronic medical records, and wherein the operations further comprise:
receiving, by the virtual assistant function, updated medical data associated with the user identity from a user associated with the user identity, second electronic medical records associated with the user identity, or a sensor or a medical device associated with the user; and
modifying the model to generate a user-specific model based on analysis, utilizing the artificial intelligence function, of the model, the updated medical data associated with the user identity, and historical medical data associated with the user identity, wherein the historical medical data comprises a portion of the first medical data, the second medical data, or the third medical data, and wherein the determining of the proposed action relating to the medical care associated with the user identity comprises determining the proposed action relating to the medical care associated with the user identity based on the user-specific model.
